(19) United States
(12) Patent Application Publication
(10) Pub. No.: US 2013/0130985 A1
(43) Pub. Date: May 23, 2013

(54) OXYTOCIN PEPTIDE ANALOGUES

(71) Applicants: Paul Alewood, Pullenvale (AU); Markus Muttenthaler, Graceville (AU); Zoltan Dekan, Taringa (AU)

(72) Inventors: Paul Alewood, Pullenvale (AU); Markus Muttenthaler, Graceville (AU); Zoltan Dekan, Taringa (AU)

(21) Appl. No.: 13/638,844

(22) PCT Filed: May 24, 2010
(86) PCT No.: PCT/AU10/00629

§ 371 Date: Nov. 16, 2012

Related U.S. Application Data
(60) Provisional application No. 61/320,049, filed on Apr. 1, 2010.

(51) Int. Cl. C07K 7/64; A61K 38/12

(52) U.S. Cl. CPC C07K 7/64
USPC 514/11.6; 530/315; 562/556; 562/553; 562/512

(57) ABSTRACT

Peptide analogues of oxytocin are described together with methods of treating conditions ameliorated by modulating oxytocin receptor. The peptide analogues are seleno or telluro peptide analogues. Methods of synthesis of selenocysteine, tellurocysteine and oxytocin analogues are also described.

The present invention relates to peptide analogues of oxytocin and their use in methods of treating conditions ameliorated by modulating the oxytocin receptor. In particular, the peptide analogues are seleno-analogues of oxytocin in which the C-terminal amide has been replaced by a carboxylic acid. The peptides of the present invention may have increased selectivity for the oxytocin receptor compared to vasopressin receptors. Methods of synthesis of selenocysteine, tellurocysteine and oxytocin analogues are also described.

Oxytocin (OT) and vasopressin (AVP, arginine-vasopressin, antidiuretic hormone) are closely related, highly conserved, multifunctional neuropeptides that are mainly synthesized in the magnocellular and parvocellular neurons of the hypothalamus. They are transported by carrier proteins, the neurophysins, into the posterior lobe of the pituitary, where they are stored. Upon stimulation they are released by enzymatic cleavage into systemic circulation, where OT is involved in uterine smooth muscle contraction during parturition, ejaculation, and milk ejection during lactation. In the central nervous system, OT functions as a neurotransmitter involved in complex social behaviors, maternal care, partnership bonding, stress and anxiety. AVP regulates peripheral fluid balance and blood pressure, and is centrally implicated in memory and learning, stress related disorders, and aggressive behaviour. In humans, OT acts via one oxytocin receptor (OTR) and AVP via three vasopressin receptors (AVPRs—vasopressor V₁ₐR, pituitary V_{1b}R, renal V₂R), which are members of the G-protein-coupled receptor family. OTRs are expressed in the brain, pituitary, kidney, ovary, testis, thymus, heart, vascular endothelium, osteoclasts, myoblasts, cardiomyocytes, pancreatic islet cells, adipocytes and several types of cancer cells. AVPRs are found in the brain, liver, kidney, vascular smooth muscle cells and most peripheral tissues.

OT and AVP are structurally very similar nonapeptides.

OT
SEQ ID NO: 1
CYIQNCPLG-NH₂
AVP
SEQ ID NO: 2
CYFQNCPRG-NH₂

The two C-terminal amidated peptides differ at positions 3 and 8. Both peptides have two cysteine residues in position 1 and 6 that form the cyclic part of the molecules followed by a 3-residue C-terminal tail.

The similarity of OT and AVP together with the high sequence homology of the extracellular binding domain of OTR and AVPRs results in significant cross talk. Specific receptor functionality is thus not controlled by ligand selectivity, but by a complex system of cell-specific up and down regulation of individual receptor expression and of the enzymes oxytocinase and vasopressinase. The ubiquitous receptor distribution and extracellular receptor homology constitutes a major hurdle in the development of therapeutics. Low receptor correlation between species complicates the problem and many compounds selective in rat are unspecific for the human receptors. Though many analogues of OT and AVP have been synthesized over the last 50 years, few receptor selective agonists and antagonists have been identified, especially for the human receptors, either for use as peptide tools or as potential therapeutics.

Clinically, OT is administered intravenously to induce labor and treat postpartum hemorrhage, and intranasally to elicit lactation. However, due to lack of selectivity, oxytocin can have multiple and severe adverse reactions in both mother and fetus. Adverse effects reported in the mother include anaphylactic reaction, postpartum hemorrhage, cardiac arrhythmia, fatal afibrinogenemia, nausea, vomiting, premature ventricular contractions, pelvic hematoma, subarachnoid hemorrhage, hypertensive episodes, rupture of the uterus, convulsions, coma and death due to water intoxication. In the neonate, adverse effects reported include bradycardia, premature ventricular contractions and other arrhythmias, jaundice, retinal hemorrhage, permanent CNS or brain damage, seizures and death. It is therefore highly desirable to develop agonists that have improved metabolic stability and/or can differentiate between the individual receptors in humans.

The present invention is predicated at least in part on the discovery that a diselenocysteine analogue of OT that has a free carboxylic acid at the C-terminus has functional selectivity for the OTR over the vasopressin receptor V₁ₐR and has enhanced metabolic stability.

In one aspect, the present invention provides a peptide of formula (I):

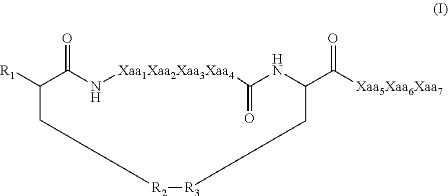

wherein R₁ is hydrogen or NH₂;

R₂ and R₃ are independently selected from —S—, —Se—, —CH₂— and —Te—, provided that R₂ and

R₃ are not both S or CH₂;

Xaa₁ is L-tyrosine, L-phenylalanine or L-tryptophan;

Xaa₂ is L-isoleucine, D-isoleucine, L-alanine, L-valine, L-leucine or L-methionine;

Xaa₃ is L-glutamine, D-glutamine or L-asparagine;

Xaa₄ is L-asparagine, D-asparagine or L-glutamine;

Xaa₅ is L-proline, D-proline, 4-hydroxyproline or 3,4-dehydroproline;

Xaa₆ is L-leucine, D-leucine, L-isoleucine, L-alanine or L-valine; and

Xaa₇ is absent, glycine, Xaa₈-Xaa₈ or a conservative substitution for glycine;

Each Xaa₈ is independently selected from glycine or a conservative substitution for glycine; and

wherein the C-terminal carboxy group is a free carboxy group (CO₂H) or is —CO₂C₁₋₁₀alkyl or —CO₂C₂₋₁₀alkenyl;
or a pharmaceutically acceptable salt thereof.

The amino acids referred to in the peptides of the invention are set out in Table 1.

TABLE 1

| Amino Acid | One letter code | Three letter code |
|---|---|---|
| L-alanine | A | Ala |
| L-asparagine | N | Asn |
| D-asparagine | n | asn |
| L-cysteine | C | Cys |
| L-glutamine | Q | Gln |
| D-glutamine | q | gln |
| glycine | G | Gly |
| L-isoleucine | I | Ile |
| D-isoleucine | i | ile |
| L-leucine | L | Leu |
| D-leucine | l | leu |
| L-methionine | M | Met |
| L-phenylalanine | F | Phe |
| L-proline | P | Pro |
| D-proline | p | pro |
| L-serine | S | Ser |
| L-threonine | T | Thr |
| L-tryptophan | W | Trp |
| L-tyrosine | Y | Tyr |
| L-valine | V | Val |
| 4-hydroxyproline | | Hyp |
| 3,4-dehydroproline | | Dhp |
| L-selenocysteine | U | Sec |
| L-tellurocysteine | | Tec |
| Sarcosine | | Sar |
| Desaminocysteine | dC | dCys |
| Desaminoselenocysteine | dU | dSec |
| Desaminotellurocysteine | | dTec |

*Desaminocysteine is also referred to as 3-mercaptopropanoic acid. Desaminoselenocysteine is also referred to as 3-selenopropanoic acid. Desaminotellurocysteine is also referred to as 3-telluropropanic acid.

As used herein, the term “conservative substitution for glycine” refers to L-alanine, sarcosine, L-3-aminopropanoic acid, L-2-methyl-3-aminopropanoic acid, L-3-methyl-3-aminopropanoic acid, L-serine, L-valine, L-leucine, L-isoleucine and L-threonine.

The term “alkyl” as used herein refers to a linear or branched hydrocarbon chain having 1 to 10 carbon atoms. Examples of suitable alkyl groups include but are not limited to methyl, ethyl, propyl, isopropyl, butyl, isobutyl, tert-butyl, pentyl, hexyl, heptyl, octyl, nonyl and decyl.

The term “alkenyl” as used herein refers to a linear or branched hydrocarbon chain having 2 to 10 carbon atoms and at least one double bond. Example of suitable alkenyl groups include, but are not limited to, ethenyl, propenyl, butenyl, 2-methylbutenyl, pentenyl, hexenyl, 2,4-hexadienyl, heptenyl, octenyl, 2,4,6-octadienyl, nonenyl and decenyl.

In some embodiments, the peptides of formula (I) may be further labelled with a radioactive fluorescent label. Fluorescent labels that may be conjugated to peptides include, but are not limited to, fluorescein, rhodamine B, fluoroscein isothiocyanate (FITC), 5-(2′-aminoethyl)-aminonaphthalene-1-sulfonic acid (EDANS), tetramethylcarboxy-rhodamine (TAMRA), Cy3, Cy5 and Alexa-fluor dyes such as Alexa-488, Alexa-546, Alexa-555, Alexa-633 and Alexa-647. Suitable radioactive labels include but are not limited to titrium (³H), carbon-14 (¹⁴C), sulfur-35 (³⁵S) and iodine-125 (I¹²⁵).

In some embodiments, one or more of the following apply:

R₂ and R₃ are both Se or Te or one of R₂ and R₃ is Se or Te and the other is S, especially where R₂ and R₃ are both Se or Te, more especially where R₂ and R₃ are both Se;

Xaa₁ is L-tyrosine or L-phenylalanine, especially L-tyrosine;

Xaa₂ is L-isoleucine, D-isoleucine or L-leucine, especially L-isoleucine;

Xaa₃ is L-glutamine or D-glutamine, especially L-glutamine;

Xaa₄ is L-asparagine or D-asparagine, especially L-asparagine;

Xaa₅ is L-proline or D-proline, especially L-proline;

Xaa₆ is L-leucine, D-leucine or L-isoleucine, especially L-leucine;

Xaa₇ is glycine, L-alanine or sarcosine, especially glycine;

the C-terminal carboxy group is CO₂H, CO₂C₁₋₄alkyl or CO₂C₂₋₄alkenyl, especially CO₂H.

In some embodiments, the peptides include amino acids which are all in the L-configuration or contain one or two amino acids in the D-configuration. In particular embodiments, the amino acids are all in the L-configuration.

In a particular embodiment, the peptide is a peptide of formula (II):

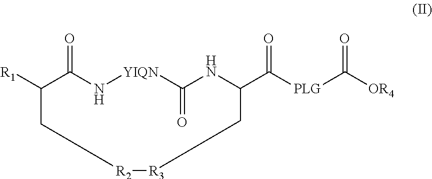

wherein R₁ is hydrogen or NH₂; and

R₂ and R₃ are independently selected from S, Se and Te, provided that both R₂ and R₃ are not S;

R₄ is H, C₁₋₁₀alkyl or C₂₋₁₀alkenyl;

or a pharmaceutically acceptable salt thereof.

The peptides of formula (I) include the following peptides:

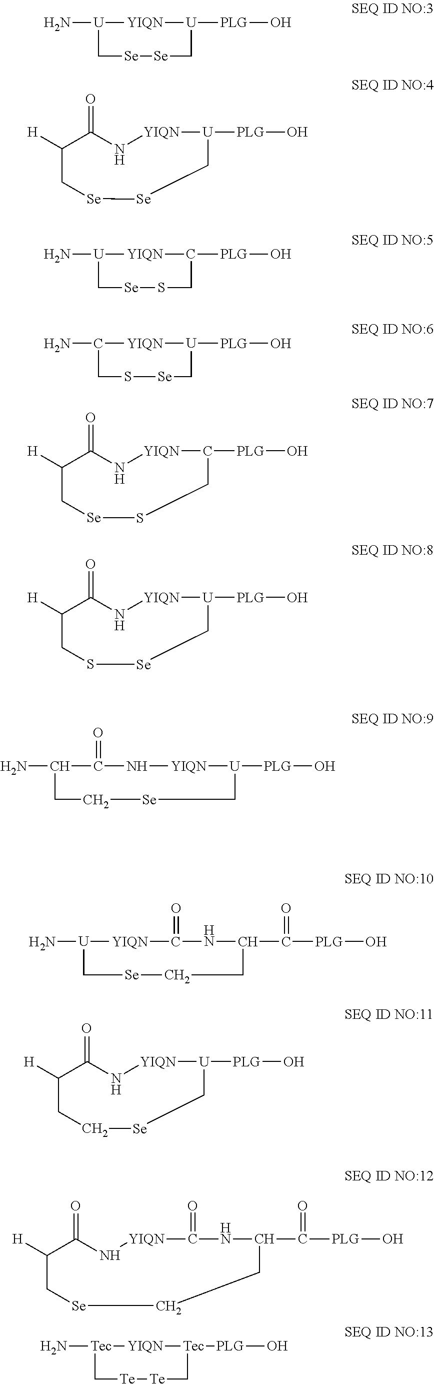

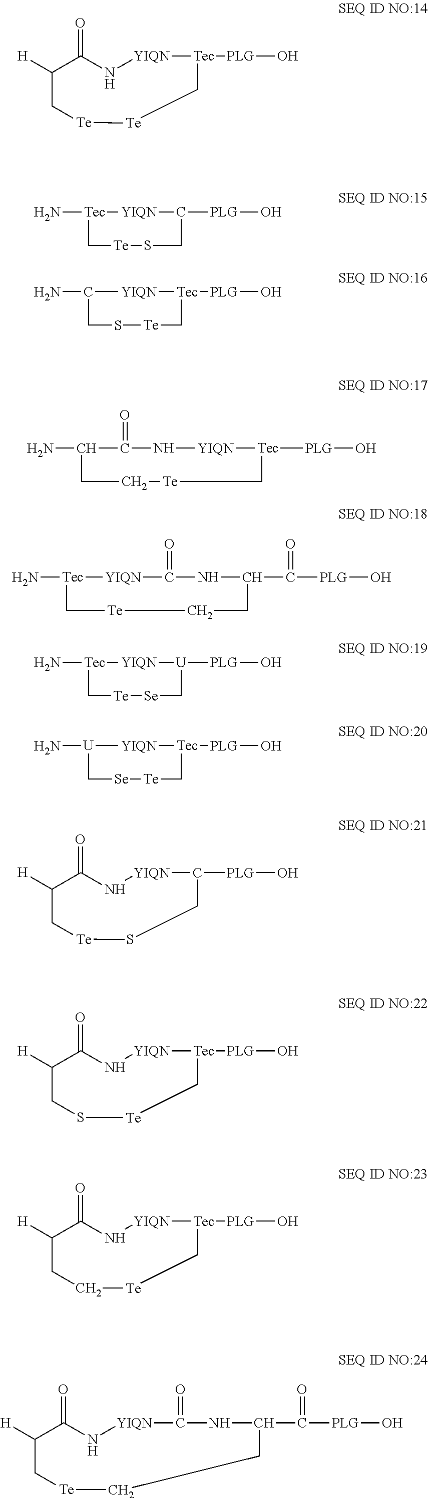

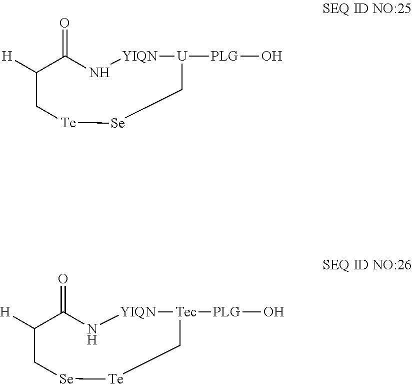

or pharmaceutically acceptable salts thereof.

In a particular embodiment, the peptide of formula (I) is a peptide of SEQ ID NO:3:

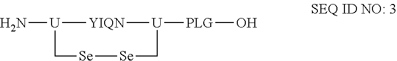

or a pharmaceutically acceptable salt thereof.

The peptides of formula (I) may be in the form of pharmaceutically acceptable salts. For example, suitable pharmaceutically acceptable salts include, but are not limited to, salts of pharmaceutically acceptable inorganic acids such as hydrochloric, sulphuric, phosphoric, nitric, carbonic, boric, sulfamic and hydrobromic acids, or salts of pharmaceutically acceptable organic acids such as acetic, propanoic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicylic, sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quaternized with such agents as lower alkyl halide, such as methyl, ethyl, propyl and butyl chlorides, bromides and iodides; dialkyl sulfates like dimethyl and diethyl sulfate; and others.

The peptides of formula (I) and formula (II) are useful in modulating the oxytocin receptor.

In one aspect, the present invention provides a method of modulating the oxytocin receptor comprising exposing the oxytocin receptor to a peptide of formula (I) or a pharmaceutically acceptable salt thereof.

In some embodiments, the OTR is located in vivo. In other embodiments, the OTR is located ex vivo. In some embodiments, the oxytocin receptor is selectively modulated. In some embodiments the peptides of formula (I) are useful as tools to identify other OTR agonists or antagonists, or agonists or antagonists of an AVR, or to study the activity of OTR. In some embodiments, the peptides of formula (I) are useful in methods of treating conditions ameliorated by modulating the OTR.

In yet another aspect, the present invention provides a method of treating a condition ameliorated by modulating the oxytocin receptor comprising administering to a subject an effective amount of a peptide of formula (I) or a pharmaceutically acceptable salt thereof.

In some embodiments, the condition ameliorated by modulating the OTR is a condition in which labor needs to be induced, a condition in which stimulation or reinforcement of uterine contractions is needed, a condition in which milk ejection during lactation is unsatisfactory, a sexual dysfunction condition such as erectile dysfunction, a condition that benefits from building or strengthening relationships such as autism, stress, depression, anxiety or an anxiety-related condition, schizophrenia or a schizophrenia-related condition, gastric disorders such as gastric inflammation, constipation and abdominal pain, some types of pain such as nociceptive pain and some types of cancer.

In some embodiments, the peptides of formula (I) and their pharmaceutically acceptable salts are capable of selectively modulating OTRs. In particular embodiments, the “modulating” is “agonizing”.

By “selectively” is meant that the peptides exert functional activity at the OTR while exerting reduced or no functional activity at AVPR, especially V₁ₐR, V_{1b}R and V₂R. In some embodiments, the peptide may bind at the OTR and AVPR but functional activity is selectively produced at the OTR. In some embodiments, the peptides of the invention have at least 5-fold selectivity for the OTR, especially at least 50-fold, 100-fold, 500-fold, 1000-fold, 2000-fold 2500-fold or greater. In particular embodiments, the selectivity is at least 2500-fold, for example 260 0-fold, 2800-fold or 3000-fold.

In some embodiments, the OTR is located ex vivo. By “ex vivo” is meant that the OTR is located in a cell or membrane preparation that is not located in a living body. The cell or membrane preparation may be used in conjunction with a peptide of formula (I) in assays that identify other selective functional agonists or antagonists of the OTR or an AVPR, especially a V₁ₐR, V_{1b}R and V₂R.

The peptides of formula (I) may also be used ex vivo or in vitro in assays to identify receptor localization in tissues and in binding or functional assays. In such assays, the peptides may be suitably labelled or conjugated with a radioactive label or with a fluorophore.

In some embodiments, the OTR is located in vivo and the peptides are used in methods of treating a condition ameliorated by modulating the OTR. In some embodiments, the condition ameliorated by modulating the OTR is a condition that requires induction of labor, a condition that requires production of uterine contractions, or a condition that requires induction of milk ejection during lactation.

In some cases it is desirable to induce labor in a pregnant woman before labor starts naturally. This may be when there are suitable reasons for concern for the mother or fetus. For example, where there are Rhesus factor problems, maternal diabetes, preeclampsia or eclampsia at or near term, after membrane rupture and for overterm babies. In some embodiments, the peptides of formula (I) or their pharmaceutically acceptable salts may be used as an adjunct therapy in the management of incomplete or inevitable abortion, particularly in the third trimester. In these cases the peptides of the invention may be effective in emptying the uterus. In some embodiments, the peptides of formula (I) or their pharmaceutically acceptable salts may be used to stimulate or reinforce uterine contractions during labor, for example, in cases of uterine inertia. The peptides of formula (I) or their pharmaceutically acceptable salts are also useful in controlling postpartum bleeding or hemorrhage.

The peptides of formula (I) or their pharmaceutically acceptable salts are also useful for treating other conditions for which exogenous OT is indicated including treatment of sexual dysfunction, depression, anxiety, anxiety-related conditions, schizophrenia, schizophrenia-related conditions, stress, some types of cancer and also for modifying complex social behaviours, particularly those involving social bonding and trust, such as encouraging maternal care, partnership building and relationship building, particularly in autism.

Oxytocin has been linked with a reduction in symptoms of anxiety and anxiety-related conditions. The peptides of formula (I) or their pharmaceutically acceptable salts are useful for reducing the symptoms of or relieving anxiety and anxiety-related conditions. Anxiety is generally considered to be a state of uneasiness or apprehension. Anxiety is considered an appropriate response to danger or threatening situations. It may also occur where there is a perceived danger or threat, also where the threat or danger is exaggerated or unfounded. Anxiety occurs in many individuals at some time in their life and can have varying cause, duration or appropriateness. However, in its serious forms, an individual may feel paralyzed from action and it may lead to serious physical or psychological effects. The term “anxiety-related conditions” includes, but is not limited to, phobias, such as agoraphobia and social phobias, acute stress disorder, post-traumatic stress disorder, generalized anxiety disorder and obsessive-compulsive disorder.

Oxytocin has also been linked to schizophrenia. Patients with schizophrenia have been shown to have decreased prepulse inhibition. Restoration of prepulse inhibition has been strongly correlated with antipsychotic drug activity. Oxytocin has been shown to dose-dependently restore prepulse inhibition induced by dizocilpine and amphetamine in a rat model. Schizophrenia is a condition that has both environmental and genetic triggers and manifests a variety of symptoms that may be positive (additional to normal behaviour) and negative (reducing normal behaviour). Positive symptoms include delusions, hallucinations, disorganised, excessive and/or repetitive speech patterns and disruptive behaviour. Negative symptoms include social withdrawal, reduced communicativeness and tonal flatness in speech. Schizophrenia may be catatonic schizophrenia, disorganized schizophrenia or paranoid schizophrenia. The term “schizophrenia-related conditions” includes disorders where at least some symptoms of schizophrenia are present but diagnosis of schizophrenia is inappropriate. For example, such conditions include, but are not limited to, schizoaffective disorder, schizophreniform disorder, delusional disorder and brief psychotic disorders. The peptides of formula (I) or their pharmaceutically acceptable salts are useful for treating the symptoms of schizophrenia and schizophrenia-related conditions.

Oxytocin is also linked to building social relationships. It is implicated in building mother-infant relationships during breast-feeding. Oxytocin has also been referred to as the “Hug Drug” where it reduces anxiety related to social interactions increasing relaxation and improving social bonding and trust. At the present time, oxytocin is being trialled for relationship building with autistic individuals. The peptides of formula (I) or their pharmaceutically acceptable salts thereof are useful for modifying social relationships; especially in autistic individuals.

Oxytocin has also been implicated in sexual function. In particular, OTRs are present in the sacral parasympathetic nucleus and the dorsal grey commissure of the L6-S1 spinal cord. Activation of these receptors causes penile erection. Stimulating oxytocinergic neurons originating in the paraventricular nucleus of the hypothalamus and extending into the extrahypothalamic brain areas controls penile erection. Oxytocin mediates erectile response to physiological stimuli such as receptive females, and maintains penile erection in response to penile stimulation. Oxytocin is also linked to stimulation of smooth muscle contraction during ejaculation. Oxytocin has also been linked to arousal, desire and orgasm and may also assist by improving social bonding between sexual partners and increasing monogamy in relationships. Oxytocin may reduce the effects of drugs that cause sexual dysfunction in males and females or may act in conjunction with other hormones to increase arousal, desire and orgasm in females. The peptides of the present invention are useful in the treatment of sexual dysfunction including erectile dysfunction and female sexual dysfunction.

It is also envisaged that the peptides of formula (I) or their pharmaceutically acceptable salts could be used in methods of enhancing normal sexual function. For example, for prolonging erection in males, increasing arousal and desire in females and intensifying orgasm in males and females.

Oxytocin receptor has been found in the gastrointestinal tract and oxytocin has been shown to improve gastric motility and reduce colonic inflammation and in some cases abdominal pain associated with constipation. The peptides of formula (I) or their pharmaceutically acceptable salts thereof are also useful in treating gastric disorders that are associated with gastric motility or colonic inflammation. Such disorders include gastroparesis, inflammatory bowel disease and irritable bowel syndrome.

Stimulation of oxytocin receptor has also been associated with relief of pain, particularly nociceptive pain. The peptides of the present invention or their pharmaceutically acceptable salts thereof may be useful in treating pain or providing analgesia in a subject. Pain relief may be achieved through modulation of OTR in the central nervous system, gut or peripheral nervous system.

Oxytocin receptor has been shown to be upregulated in some cancer cells and to inhibit cell proliferation and growth in some human carcinoma cells (Strunecká et al. Folia Biologica (Praha) 55, 159-165 (2009)). The peptides of the invention are useful in treating tumorous cancer, especially ovarian carcinoma, endometrial carcinoma, breast cancer, prostatic stromal cell cancer, neuroblastoma and glioblastoma. The peptides of the present invention may be used to deliver a chemotherapeutic agent or radioactive agent to a tumor cell that over-expresses OTR or to deliver a label or contrast agent that may assist in surgical removal of a tumor, or to identify the presence of a tumor or a metastatic tumor.

In one embodiment there is provided a method of inducing labor or improving uterine contractions in a pregnant woman comprising administering an effective amount of a peptide of formula (I) or a pharmaceutically acceptable salt thereof.

In another embodiment, there is provided a method of inducing milk ejection in a lactating woman comprising administering an effective amount of a peptide of formula (I) or a pharmaceutically acceptable salt thereof.

In yet another embodiment, there is provided a method of inducing treating or preventing sexual dysfunction in a subject comprising administering an effective amount of a peptide of formula (I) or a pharmaceutically acceptable salt thereof.

In yet another embodiment, there is provided a method of treating or preventing anxiety, anxiety-related conditions, stress, schizophrenia, schizophrenia-related conditions, or for modifying complex social behaviours in a subject comprising administering an effective amount of a peptide of formula (I) or a pharmaceutically acceptable salt thereof.

In a further embodiment, there is provided a method of enhancing normal sexual function in a subject comprising administering an effective amount of a peptide of formula (I) or a pharmaceutically acceptable salt thereof.

In a further embodiment, there is provided a method of treating or preventing gastrointestinal disorders comprising administering an effective amount of a peptide of formula (I) or a pharmaceutically acceptable salt thereof.

In another embodiment, there is provided a method of treating or preventing pain in a subject comprising administering an effective amount of a peptide of formula (I) or a pharmaceutically acceptable salt thereof.

In yet a further embodiment, there is provided a method of treating or diagnosing a cancer in which the OTR is over-expressed comprising administering an effective amount of a peptide of formula (I) or a pharmaceutically acceptable salt thereof.

The peptides of formula (I) or their pharmaceutically acceptable salts may be useful for treating any mammalian subject including humans, primates, livestock animals (e.g.: sheep, cattle, pigs, horses), laboratory test animals (e.g.: mice, rats, rabbits, guinea pigs), companion animals (e.g.: cats and dogs), especially humans.

In another aspect of the invention, there is provided a use of a peptide of formula (I) or a pharmaceutically acceptable salt thereof in the manufacture of a medicament for treating a condition ameliorated by modulating the OTR.

In yet another aspect of the invention there is provided a use of a peptide of formula (I) or a pharmaceutically acceptable salt thereof in therapy, especially therapy of a condition ameliorated by modulating the OTR.

Although it is possible that the peptides of the invention are used without additives, it is more likely that they are formulated into a composition for use. In a further aspect of the invention there is provided a pharmaceutical composition comprising a peptide of formula (I) or a pharmaceutically acceptable salt thereof and a pharmaceutically acceptable carrier or excipient. In particular embodiments, the pharmaceutical composition comprises a peptide of formula (II) such as a peptide of SEQ ID NO:3 or a pharmaceutically acceptable salt thereof.

As will be readily appreciated by those skilled in the art, the route of administration and the nature of the pharmaceutically acceptable carrier will depend on the nature of the condition and the mammal to be treated. It is believed that the choice of a particular carrier or delivery system and the route of administration could be readily determined by a person skilled in the art. In the preparation of any formulation containing peptide actives care should be taken to ensure that the activity of the peptide is not destroyed in the process and that the peptide is able to reach its site of action without being destroyed. In some circumstances, it may be necessary to protect the peptide by means known in the art, such as, for example, micro encapsulation. Similarly the route of administration chosen should be such that the peptide reaches its site of action. In view of the improved stability of the peptides of formula (I) relative to native OT, a wider range of formulation types and routes of administration are available.

The pharmaceutical forms suitable for injectable use include sterile injectable solutions or dispersions, and sterile powders for the extemporaneous preparation of sterile injectable solutions. They should be stable under the conditions of manufacture and storage and may be preserved against the contaminating action of microorganisms such as bacteria and fungi. The solvent or dispersion medium for the injectable solution or dispersion may contain any of the conventional solvent or carrier systems for peptide actives, and may contain, for example, water, ethanol, polyol (for example, glycerol, propylene glycol and liquid polyethylene glycol and the like) and electrolytes, and mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about where necessary by the inclusion of various antibacterial and antifungal agents, for example, parabens, chlorobutanol, phenol, sorbic acid, thimerosal, and the like. In many cases it will preferable to include agents to adjust osmolality, for example, sugars or sodium chloride. In some embodiments, the formulation for injection will be isotonic with blood. Prolonged absorption of the injectable compositions can be brought about by the use in the compositions of agents delaying absorption, for example, aluminium monostearate and gelatine. Pharmaceutical forms suitable for injectable use may be delivered by any appropriate route including intravenous, intramuscular, intracerebral, intrathecal injection or by infusion.

Sterile injectable solutions are prepared by incorporating the peptides in the required amount in the appropriate solvent with various of the other ingredients enumerated above, as required, followed by filtered sterilization. Generally, dispersions are prepared by incorporating the various sterilized components into a sterile vehicle which contains the basic dispersion medium and the required other ingredients from those enumerated above. In the case of sterile powders for the preparation of sterile injectable solutions, the methods of preparation are known in the art, particularly vacuum drying and the freeze-drying technique which yield a powder of the peptide plus any additional desired ingredient from previously sterile-filtered solution thereof.

The peptides of the present invention may be orally administered, for example, with an inert diluent or with an assimilable edible carrier, or it may be enclosed in a hard or soft shell gelatine capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers and the like. Such compositions and preparations preferably contain at least 1% by weight of the peptide. The percentage of the compositions and preparations may, of course, be varied and may conveniently be between about 5 to about 80% of the weight of the dosage unit. The amount of peptide in such therapeutically useful compositions is such that a suitable dosage will be obtained.

The tablets, troches, pills, capsules and the like may also contain the components as listed hereafter: a binder such as gum, acacia, corn starch or gelatine; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills or capsules may be coated with shellac, sugar or both. A syrup or elixir may contain the active compound, sucrose as a sweetening agent, methyl and propylparabens as preservatives, a dye and a flavouring such as cherry or orange flavour.

Of course, any material used in preparing dosage unit form should be pharmaceutically pure and substantially non-toxic in the amounts employed. In addition, the peptides may be incorporated into sustained-release preparations and formulations.

The present invention also extends to other forms of administration, for example, topical application, such as creams, lotions and gels, and compositions suitable for inhalation or intranasal delivery, for example solutions or dry powders.

In some embodiments, intranasal or inhalation administration is used. Solutions or suspensions may be applied directly to the nasal cavity by conventional means, for example, with a dropper, pipette or spray. The formulations may be provided in single dose or multidose form. In the latter case of a dropper or pipette, this may be achieved by the subject administering an appropriate, predetermined volume of solution or suspension. In the case of a spray, this may be achieved for example, by means of a metering atomising spray pump. To improve nasal delivery and retention, the peptides may be encapsulated with cyclodextrins, or formulated with agents expected to enhance delivery and retention in the nasal mucosa.

Administration to the respiratory tract may also be achieved by means of an aerosol formulation in which the peptide or its pharmaceutically acceptable salt is provided in a pressurized pack with a suitable propellant such as a chlorofluorocarbon (CFC), for example, dichlorodifluoromethane, trichlorofluoromethane or dichlorotetrafluoroethane, carbon dioxide or other suitable gas. The aerosol may conveniently also contain a surfactant such as lecithin. The dose of drug may be controlled by provision of a metered valve.

Alternatively the peptide or its pharmaceutically acceptable salt may be provided in the form of a dry powder, for example, a powder mix of the peptide in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form, for example in capsules or cartridges of, for example, gelatine, or in blister packs from which the powder may be administered by means of an inhaler. In formulations intended for administration to the respiratory tract, including intranasal formulations, the peptide or its pharmaceutically acceptable salt will generally have a small particle size, for example, in the order of 1 to 10 microns or less. Such a particle size may be obtained by means known in the art, such as micronization.

In particular embodiments, parenteral dosage forms such as those for intravenous or intramuscular delivery are used, especially by intravenous infusion. These dosage forms are particularly useful in stimulating uterine contractions. In some embodiments, formulations for intranasal delivery are preferred, such as those for use during lactation or those for treating sexual dysfunction, stress, anxiety or for building relationships.

An effective amount of the peptides of formula (I) or their pharmaceutically acceptable salts is one that achieves or partly achieves the result desired and will depend on the health and physical condition of the individual being treated, the formulation and administration route of the formulation and the medical situation. It is expected that the amount will fall within a relatively broad range that can be determined by routine trial.

An effective dose of the peptides of formula (I) will be in the range of about 10 to about 100 International Units (I.U.) for example 10 to 50 I.U. When administration is by intravenous infusion for inducing labor or reinforcing uterine contractions in a pregnant woman, an infusion of 0.5 to 2.0 mU/min, especially 0.5 to 1.0 mU/min is used then increased incrementally every 30 to 60 minutes by 1 to 4 mU/min, especially 1 to 2 mU/min until the desired frequency and strength of contraction is obtained. The dosage may then be reduced incrementally in a similar manner. In some cases, the peptide of formula (I) may be delivered by intramuscular injection to increase muscle contraction.

For postpartum uterine bleeding, an intravenous infusion of 10 to 80 units, especially 10 to 40 units, may be used to sustain uterine contraction. Alternatively intramuscular injection of about 10 to 20 units, especially 10 units may be used after delivery of the placenta.

In incomplete or inevitable abortion, an intravenous infusion of about 10 to 20, especially 10 units in 500 mL may be used.

Intranasal delivery of a peptide of formula (I) or its pharmaceutically acceptable salt is suitable for lactating mothers, for sexual dysfunction and for behavioural effects such as relationship building and for treating stress, anxiety, anxiety-related conditions, schizophrenia and schizophrenia-related conditions. Suitable doses include about 10 to about 70 I.U. of the peptide of formula (I) or its pharmaceutically acceptable salt; especially about 10 to 60 I.U., 10 to 35 I.U. or 20 to 30 I.U. per dose.

The peptides of the invention or their pharmaceutically acceptable salts may be administered in association with other pharmaceutical agents that assist in labor or lactation or building relationships or are used to treat sexual dysfunction, anxiety or stress. By “in association with” is meant that the peptide and the other agent are administered together in a single composition or are administered in separate compositions in a manner that allows the two compounds to be biologically active at the same time. Examples of other agents that may be administered in association with the peptides of the present invention include oestrogen for male sexual dysfunction, and ergometrine or its salts such as ergometrine maleate, during labor.

The peptides of formula (I) may be prepared by methods known in the art such as solution phase or solid phase peptide synthesis, especially solid phase peptide synthesis (SPPS). The synthesis may use t-butoxycarbonyl (Boc)-protected or 9-fluorenylmethoxycarbonyl (Fmoc)-protected amino acids and activation, deprotection and coupling steps as are well known in the art. To produce C-terminal carboxylic acid, the resin used in SPPS is one that produces a carboxylic acid upon cleavage, for example, N^{α}-Boc-L-amino acid-phenylacetamido-methyl (PAM) resin.

The peptides may be produced by substituting one or both cysteine residues of OT with a protected selenocysteine or tellurocysteine residue which after completion of the peptide synthesis, deprotection and cleavage, will be oxidized to provide a diselenide bond, ditelluride bond, a telluroseleno bond, a tellurosulfide bond or a selenosulfide bond. In another embodiment, the N-terminal cysteine residue may be substituted by a 3-mercaptopropanoic acid residue, a 3-selenopropanoic acid residue or a 3-telluropropanoic acid residue to provide the desamino-OT analogues (R₁ is H). In yet another embodiment, one of the selenium atoms in the diselenide bond or one of the tellurium atoms is replaced by a —CH₂— group. This embodiment provides a long acting oxytocin receptor agonist.

In another embodiment, a peptide comprising a cyclic selenoether or telluroether may be prepared in an analogous manner to means of preparing cyclic thioethers. For example, the peptide may be prepared using Fmoc SPPS and incorporate a 2-amino-4-chlorobutanoic acid residue at the 1 or 6 position or a 4-chlorobutanoic acid residue at the 1 position. After synthesis the cyclic peptide is formed by the reaction of the seleno or telluro group and the —CH₂—Cl group of the butanoic acid.

Suitable protecting groups for the sulphur of cysteine, the seleno group of selenocysteine or the telluro group of tellurocysteine include, but are not limited to, acetamidomethyl (Acm), p-nitrobenzyl (pNB), o-nitrobenzyl (oNB), 4-methoxybenzyl or 4-methylbenzyl. In some embodiments using Boc-SPPS, a 4-methylbenzyl protecting group is favoured for sulfur, seleno or telluro protection. In other embodiments using Fmoc-SPPS, a 4-methoxybenzyl protecting group is favoured.

Selenocysteine may be obtained by a number of known methods. For example, seleno-L-cystine may be purchased and treated with a reducing agent such a sodium borohydride and then reacted with an activated protecting group to provide a protected seleno-cysteine group as shown in scheme 1:

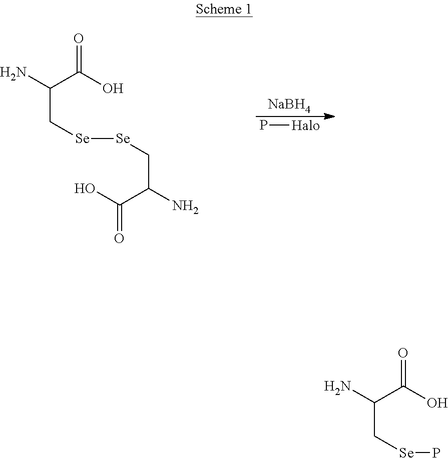

wherein P is a protecting group and Halo is a halogen atom especially chloro, bromo or iodo, or a similar leaving group. In this synthesis, protection of the amino or carboxy functionality may be required or subsequently, the amino group may be protected with Boc or Fmoc ready for use in SPPS.

In another method, β-chloroalanine is treated with a combination of sodium borohydride and metallic selenium which forms disodium diselenide in situ, to produce seleno-L-cystine as shown in scheme 2:

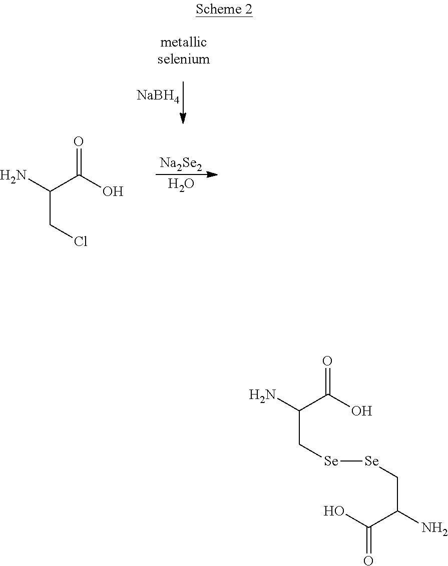

A protected selenocysteine can then be prepared from the seleno-L-cystine as shown in scheme 1. This synthetic pathway may be readily adapted to produce tellurocysteine compounds, with substitution of metallic selenium with metallic tellurium.

Again the amino group may be further protected ready for SPPS if required. For example, Boc protection may be introduced by reaction of the protected selenocysteine or tellurocysteine with Boc₂O and K₂CO₃ or Fmoc protection may be introduced by reaction of the protected selenocysteine with Fmoc-OSu in the presence of triethylamine (TEA), acetonitrile and water.

Suitable protection and deprotection strategies for reactive groups found in peptides and which are useful in SPPS can be found in Green & Wuts, Protective Groups in Organic Synthesis, Third Edition, 1999, John Wiley & Sons.

A number of methods of synthesis of selenocysteine compounds are described in Muttenthaler and Alewood, 2008, J. Peptide Sci., 14:1223-1239.

Many of the methods for preparing selenocysteine derivatives are time intensive and require a number of steps including amino and/or carboxy protection and/or deprotection. Some methods also produce toxic H₂Se gas. There is a need for new synthetic routes for preparing protected selenocysteine compounds for use in peptide synthesis that are less toxic, are less laborious and have higher yields.

In one aspect of the present invention there is provided a method of preparing a Se-protected selenocysteine or Te-protected tellurocysteine, suitable for use in peptide synthesis; said method comprising:
i) treating an alanine derivative of formula (III):

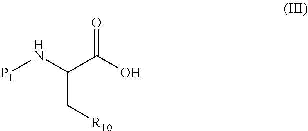

wherein P₁ is hydrogen or an amino protecting group and R₁₀ is a leaving group;
with a diselenide salt prepared from combining metallic selenium or tellurium and a first reducing agent in an anhydrous solvent, to produce a diselenide or ditelluride of formula (IV):

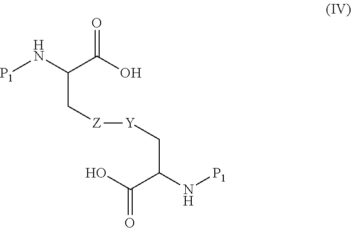

in which Z and Y are Se or Te;
ii) treating the diselenide of formula (IV) with a second reducing agent and an activated protecting group to provide a Se-protected selenocysteine or Te-protected tellurocysteine of formula (V):

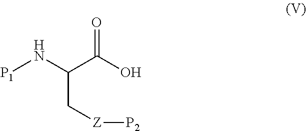

wherein Z is Se or Te, P₁ is hydrogen or an amino protecting group and P₂ is a seleno- or telluro-protecting group.

P₁ may be hydrogen and then after the selenocysteine or tellurocysteine has been prepared the amino nitrogen may be protected by a protecting group suitable for use in SPPS, for example Boc or Fmoc. Alternatively, the amino group may be protected before synthesis of the selenocysteine or tellurocysteine so that it is ready for use in SPPS. Optionally the seleno- or telluro-protecting group can be removed.

In some embodiments, the amino protecting group, P₁, is tert-butyloxycarbonyl (Boc). In some embodiments, the activated seleno- or telluro-protecting group is selected from 4-methylbenzylchloride, acetamidomethanol, p-nitrobenzylchloride or o-nitrobenzylchloride, especially p-nitrobenzylchloride or 4-methylbenzylchloride. In some embodiments, the seleno-protecting group is 4-methylbenzyl, acetamidomethyl, p-nitrobenzyl or o-nitrobenzyl, especially p-nitrobenzyl or 4-methylbenzyl.

R₁₀ is a facile leaving group such as a halogen, for example, F, Cl, Br or I, especially Cl, Br or I, more especially chlorine.

In some embodiments, the first reducing agent is an agent able to reduce selenium or tellurium in an anhydrous solvent. Examples of suitable reducing agents include hydride reagents and borohydride reagents such as sodium borohydride (NaBH₄), lithium aluminium hydride (LiAlH₄), lithium borohydride (LiBH₄), potassium borohydride (KBH₄), lithium (triethyl) borohydride and diisobutylaluminium hydride (DIBALH); hydrazines such as hydrazine and phenylhydrazine; and samarium reducing agents such as samarium iodide (SmI₂). In particular embodiments, the reducing agent is lithium(triethyl)borohydride.

In some embodiments, the second reducing agent is the same or different from the first reducing agent and may be selected from hydride reagents and borohydride reagents such as sodium borohydride (NaBH₄), lithium aluminium hydride (LiAlH₄), lithium borohydride (LiBH₄), potassium borohydride (KBH₄), lithium (triethyl)borohydride and diisobutylaluminium hydride (DIBALH); hydrazines such as hydrazine and phenylhydrazine; and samarium reducing agents such as samarium iodide (SmI₂). In particular embodiments, the second reducing agent is sodium borohydride.

The first reduction is performed in an anhydrous solvent to reduce or eliminate the H₂Se or H₂Te gaseous byproduct. Suitable solvents include tetrahydrofuran (THF), dichloromethane, diethylether and dimethylformamide, especially anhydrous THF.

In yet another aspect of the present invention, there is provided a method of preparing seleno- or telluro-protected desaminocysteine acid comprising the steps of:
i) treating a propanoic acid having a 3-position leaving group, R₁₀, with nucleophilic selenium or tellurium and to produce a diselenide or ditelluride dimer

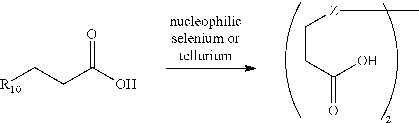

in which Z is Se or Te;
and
ii) reducing the diselenide or ditelluride dimer in the presence of an activated protecting group to obtain the protected 3-selenopropanoic acid or protected 3-telluropropanoic acid.
iii)

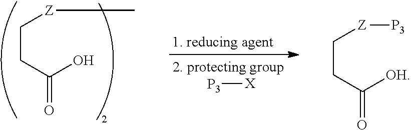

The terms 3-selenopropanoic acid and desaminoselenocysteine or 3-telluropropanoic acid and desaminotellurocysteine are used interchangeably.

R₁₀ is a facile leaving group such as a halogen, for example, F, Cl, Br, I especially Cl, Br or I.

The nucleophilic selenium or tellurium may be in any suitable form to displace the leaving group R₁₀. For example, the nucleophilic selenium or tellurium may be disodium diselenide/disodium ditelluride, (Na₂Se₂/Na₂Te₂) in a solvent such as water or dilithium diselenide/dilithium ditelluride (Li₂Se₂/Li₂Te₂) in a solvent such as THF. Alternatively, the nucleophilic selenium or tellurium may be produced in situ with LiAlHSeH.

The reducing agent step (ii) may be selected from hydride reagents and borohydride reagents such as sodium borohydride (NaBH₄), lithium aluminium hydride (LiAlH₄), lithium borohydride (LiBH₄), potassium borohydride (KBH₄), lithium(triethyl)borohydride and diisobutylaluminium hydride (DIBALH); hydrazines such as hydrazine and phenylhydrazine; and samarium reducing agents such as samarium iodide (SmI₂). In particular embodiments, the reducing agent is sodium borohydride.

In some embodiments, the activated seleno- or telluro-protecting group, P₃ is selected from 4-methylbenzylchloride, acetamidomethanol, p-nitrobenzylchloride or o-nitrobenzylchloride, especially p-nitrobenzylchloride or 4-methylbenzylchloride. In some embodiments, the seleno- or telluro-protecting group is 4-methylbenzyl, acetamidomethyl, p-nitrobenzyl or o-nitrobenzyl, especially p-nitrobenzyl or 4-methylbenzyl.

P₃ can be removed after the 3-seleno-propanoic acid or 3-telluro-propanoic acid is incorporated at the N-terminus of the peptide and oxidized to provide a diselenide or selenosulfide bond in the peptide of the invention.

In another method of preparing oxytocin analogues, the peptide may be prepared by solid phase synthesis where the cysteine residues are replaced by serine residues or β-chloroalanine residues. If serine residues are used, they are subsequently converted to β-chloroalanine residues. The peptide may then be treated with nucleophilic selenium (e.g.: Na₂Se) or tellurium (e.g.: Na₂Te) followed by acidification, for example with trifluoroacetic acid. This method is particularly suitable for preparing ditelluro-OT analogues.

In one aspect of the invention there is provided a method of preparing a diseleno or ditelluro oxytocin peptide analogue comprising:
i) preparing an oxytocin peptide analogue of formula (VI) on a solid phase synthesis resin:

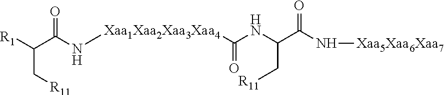

Xaa₁ is L-tyrosine, L-phenylalanine or L-tryptophan;

Xaa₂ is L-isoleucine, D-isoleucine, L-alanine, L-valine, L-leucine or L-methionine;

Xaa₃ is L-glutamine, D-glutamine or L-asparagine;

Xaa₄ is L-asparagine, D-asparagine or L-glutamine;

Xaa₅ is L-proline, D-proline, 4-hydroxyproline or 3,4-dehydroproline;

Xaa₆ is L-leucine, D-leucine, L-isoleucine, L-alanine or L-valine; and

Xaa₇ is absent, glycine, Xaa₈-Xaa₈ or a conservative substitution for glycine;

wherein each R₁₀ is independently selected from Cl, B or I;
ii) treating the peptide with neucleophilic selenium or tellurium to produce a peptide of the formula (VII)

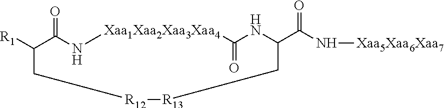

wherein R₁ and Xaa₁ to Xaa₇ are as defined above and R₁₂ and R₁₃ are both Se or Te; and
iii) cleaving the peptide from the solid phase synthesis resin.

The peptide is synthesised by solid phase peptide synthesis methods.

In some embodiments, each R₁₁ is chloro. In some embodiments, the peptide is synthesised using serine residues to provide the amino acids bearing R₁₁ and the serine hydroxy is converted to a halo group such as chloro, after addition of the serine in the peptide synthesis. Methods of converting a hydroxy group to a halo group, are known in the art. For example, a serine hydroxy may be replaced by a chloro group by treatment with Ph₃P and Cl₃CN.

In some embodiments the nucleophilic selenium or tellurium is H₂Se₂ or H₂Te₂.

After synthesis, the cyclised peptide may be deprotected and/or removed from resin using methods known in the art. In some embodiments, cleavage may produce a C-terminal amide or a C-terminal carboxylic acid, especially a C-terminal carboxylic acid. Esters may be produced by methods known in the art for esterification of a carboxylic acid.

The invention will now be described with reference to the following Figures and examples which illustrate some of the preferred aspects of the present invention. However, it is to be understood that the particularity of the following description of the invention is not to supersede the generality of the preceding description of the invention.

### BRIEF DESCRIPTION OF FIGURE

FIG. 1 provides functional selectivity profile of SEQ NO:1 Oxytocin and SEQ NO:3 [C1,6U]-OT-OH over all four OT/AVP receptors. [C1,6U]-OT-OH displayed at least (depending on the assay) a 1200-fold selectivity improvement for the hOTR over the hV₁ₐR and a 120-fold selectivity improvement over the hV_{1b}R and hV₂R compared to oxytocin.

### EXAMPLES

### Materials

N^{α}-Boc- and N^{α}-Fmoc-L-amino acids, Fmoc-Rink amide 4-methylbenzhydrylamine (MBHA) resin and reagents used during chain assembly were peptide synthesis grade purchased from Novabiochem (Merck Pty., Kilsyth, Vic., Australia) and Bachem (Bubendorf, Switzerland). N^{α}-Boc-L-amino acid-phenylacetamidomethyl (Pam)-resin was purchased from Peptides International (Louisville, Ky., USA), MBHA resin from Peptide Institute (Osaka, Japan). [2-(1H-benzotriazol-1-yl)-1,1,3,3-tetramethyluronium hexa-fluorophosphate] (HBTU) and 7-azabenzotriazol-1-yloxy-tris-(pyrrolidino)phosphonium hexafluoro-phosphate (PyAOP) were purchased from Fluka (Buchs, Switzerland), 2-(1H-7-azabenzotriazol-1-yl)-1,1,3,3-tetramethyl uronium hexafluorophosphate (HATU) from GenScript Corporation (Piscataway, N.J., USA), N,N′-diisopropylethylamine (DIEA), trifluoroacetic acid (TFA), dichloromethane (DCM) and N,N′-dimethylformamide (DMF) from Auspep (Melbourne, Vic., Australia). Anhydrous hydrogen fluoride (HF) was purchased from. BOC Gases (Sydney, NSW, Australia) and p-cresol and p-thio-cresol, as well as all other organic reagents and solvents, unless stated otherwise, were purchased in their highest purity from Sigma-Aldrich (Sydney, NSW, Australia). All solvents for solid-phase peptide synthesis (SPPS) were of peptide synthesis grade and used without further purification. HPLC grade acetonitrile (Lab Scan, Bangkok, Thailand) and water measuring 18.2 MΩ (ELGA, Melbourne, Vic., Australia) were used for the preparation of all solvents for liquid chromatography.

### Example 1

### N^{α}-tert-butyloxycarbonyl-Se(acetamidomethyl)-L-selenocysteine; Boc-L-Sec(Acm)-OH

Sodium borohydride (NaBH₄, 0.57 g, 15 mmol) was added to a solution of L-selenocysteine (2 g, 6 mmol) in 50 mL 0.1 M NH₄HCO₃ pH 9 at 0° C. and under argon atmosphere. After 15 min the mixture was allowed to warm to room temperature and stirred until colorless. After addition of 20 mL of dioxane the solution was cooled to 0° C. and 6 M HCl was added drop-wise until pH 1-2 was reached. Acetamidomethanol (1.3 g, 14.4 mmol) was added and the reaction mixture was stirred over night. Dioxane was removed in vacuo and the residue was lyophilized. The crude residue was redissolved in 20 mL dioxane and 20 mL water, and cooled to 0° C. followed by addition of K₂CO₃ (3.31 g, 24 mmol) (pH 9-10). While keeping the reaction mixture at 0° C., di-tert-butyl dicarbonate (2.9 g, 13.2 mmol) was added, the solution was allowed to reach room temperature within 30 min and it was stirred for another 3 h. Dioxane was removed in vacuo, the solution was diluted with 30 mL ether and 30 mL water, the layers separated and the aqueous layer was washed a second time with ether. The aqueous solution was acidified with solid citric acid to a pH of 2-3, and then extracted three times with ethyl acetate (EtOAc), washed with 10% citric acid and brine, dried over MgSO₄, evaporated, chased with ether and dried over high vacuum. The resulting light yellow oil was recrystallized in ether/petroleum ether, yielding 1.3 g N^{α}-tert-butyloxycarbonyl-acetamidomethyl-L-selenocysteine (32% yield, >98% purity by HPLC). MS [m/z]: MS [calc.]: 341.0615 [M+H]; MS [found]: 341.0607 [M+H], 285.0242 [M-tBu], 241.0176 [M-tBoc]. m.p. 141-143° C. ¹H-NMR, (600 MHz, CDCl₃), δ [ppm]: 1.46 (9H, s), 2.05 (3H, s), 3.05 (1H, dd, βCH), 3.20 (1H, dd, βCH), 4.53 (2H, d, Se—CH₂—NH), 4.61 (1H, m, αCH), 5.64 (1H, d, CO—NH—CH), 7.01 (1H, t, Se—CH₂—NH). ¹³C-Apt (150 MHz, CDCl₃) δ [ppm]: 23.3 (1CH₃, CO—CH₃), 26.9 (1CH₂, βCH₂), 28.5 (3CH₃), 33.6 (1CH₂, Se—CH₂—NH), 54.0 (1CH, αCH), 156.2 (1C, O—CO—NH), 172 (1C, NH—CO—CH₃), 173.6 (1C, COOH).

### Example 2

### N^{α}-tert-butyloxycarbonyl-4-nitrobenzyl-L-selenocysteine (Boc-L-Sec(pNB)-OH)

Triethylborohydride (LiB(Et)₃H, Super Hydride, 1 M in anhydrous THF, 76.2 mL, 76.2 mmol) was added drop-wise to metallic selenium (5.7 g, 72.6 mmol) in 40 mL anhydrous THF under nitrogen atmosphere. The suspension was heated to reflux and stirred for 1 h. Boc-β-chloro-L-alanine (5 g, 22.4 mmol) was dissolved in 30 mL anhydrous THF in a separate three-necked round bottom flask. The solution was cooled to −78° C. and the dilithium diselenide solution was transferred under nitrogen atmosphere into the solution via a cannula. The mixture was kept under nitrogen atmosphere, stirred at −78° C. for 15 min, was then allowed to warm to room temperature and stirred over night. The solution was filtered through filter-agent-celite, which was then washed with 20% MeOH/EtOAc (v/v) to ensure the product was removed. Solvent evaporation in vacuo yielded 4.2 g of crude Boc-L-selenocystine, which was used without further purification. It was redissolved in 40 mL ethanol, cooled to 0° C. and purged with argon. NaBH₄ (2.1 g, 55 mmol) was added at 0° C., and the reaction was kept under argon. After 30 min the mixture was allowed to warm to room temperature and stirring continued until the solution turned colorless. α-Bromo-p-nitrobenzyl (4.1 g, 18.9 mmol) in 40 mL THF was added via a dropping funnel over a period of 30 min. The mixture was stirred for another 2 h at room temperature. After evaporation of THF in vacuo 200 mL of 50% ether and 50% water were added to the remaining solution. The layers were separated and the aqueous layer washed a second time with ether. The aqueous solution was acidified with solid citric acid to pH 2-3, extracted 3× with EtOAc, washed with 10% citric acid and brine, dried over anhydrous MgSO₄, filtered and evaporated in vacuo. Purification by column chromatography (DCM:i-ProH:AcOH, 97.5:2.0:0.5) yielded 4.7 g of brown amorphous N^{α}-tert-butyloxycarbonyl-4-nitrobenzyl-L-selenocysteine (52% yield, >98% purity by HPLC). MS [calc.]: 405.0565 [M+1]; MS [found]: 405.0557 [M+1], 349.0124 [M-tBu], 305.1278 [M-tBoc]. ¹H-NMR (400 MHz, CDCl₃) δ [ppm]: 1.43 (s, 9H, CH₃ Boc); 2.92 (dd, 1H, βCH); 3.00 (dd, 1H, βCH); 3.88 (s, 2H, CH₂ pNB); 4.63-4.65 (m, 1H, αCH); 5.33 (d, 1H, NH); 7.45 (d, 2H, aromatic); 8.15 (d, 2H, aromatic) ¹³C-NMR (150 MHz, CDCl₃) δ [ppm]: 26.1 (βCH₂); 27.1 (CH₂ pNB); 28.5 (CH₃ Boc); 53.5 (αCH); 81.1 (C Boc); 124.1 (CH aromatic); 129.9 (CH aromatic); 146.9 (2×C aromatic); 155.6 (C═O Boc); 175.4 (COOH).

### Example 3

### N^{α}-tert-butyloxycarbonyl-Se(para-nitrobenzyl)-L-selenocysteine (Boc-L-Sec(pNB)-OH) (Alternative Synthesis)

3.2 g (83.8 mmol) of NaBH₄ was added in two portions to a solution of 4 g (12 mmol) of L-selenocysteine, dissolved in 40 mL 1 M NaOH under argon atmosphere and at 0° C. The pH was adjusted to 10 by addition of 1 M HCl. The ice-water bath was removed after 15 min and the solution stirred until colorless. 5.7 g (26.3 mmol) of p-nitrobenzyl bromide dissolved in 30 mL THF was added drop-wise over 1 h at 0° C. The solution was stirred another 4 h at 0° C. before the product was precipitated as a white hydrochloride by addition of 6 M HCl. It was filtered, washed with cold ether and dried over night in vacuo over phosphorous pentoxide. 8.1 g (23.9 mmol) of Se-(methyl-para-nitrobenzyl)-L-selenocysteine hydrochloride was obtained (99% yield), which was dissolved in 110 mL of water and 55 mL of dioxane. 6.62 g (47.9 mmol) of potassium carbonate was added to the solution and the mixture was heated gently until everything dissolved. After cooling on a water-ice bath 6.27 g (28.73 mmol) di-tert-butyl dicarbonate was dissolved in 55 mL of dioxane and added slowly to the reaction mixture. The solution was allowed to come to room temperature within 30 min and was stirred for another 2 h. Dioxane was removed in vacuo and 50 mL of 50% ether and 50% water was added to the remaining solution. The layers were separated and the aqueous layer washed a second time with ether. The aqueous solution was acidified with solid citric acid, extracted three times with ethyl acetate, washed with 10% citric acid and brine, dried over anhydrous MgSO₄, filtered and evaporated in vacuo. Purification by column chromatography (DCM:i-ProH:AcOH, 97.5:2.0:0.5) yielded 3.5 g (8.9 mmol) of brown amorphous N^{α}-tert-butyloxycarbonyl-4-nitrobenzyl-L-selenocysteine (37% yield, >98% purity by HPLC). MS [m/z]: MS [calc.]: 405.0565 [M+1]; MS [found]: 405.0557 [M+1], 349.0124 [M-tBu], 305.1278 [M-tBoc]. ¹H-NMR (400 MHz, CDCl₃) δ [ppm]: 1.43 (s, 9H, CH₃ Boc); 2.92 (dd, 1H, βCH); 3.00 (dd, 1H, βCH); 3.88 (s, 2H, CH₂ pNB); 4.63-4.65 (m, 1H, αCH); 5.33 (d, 1H, NH); 7.45 (d, 2H, aromatic); 8.15 (d, 2H, aromatic). ¹H-NMR (400 MHz, DMSO) δ [ppm]: 1.36 (s, 9H, CH₃ Boc); 2.70 (dd, 1H, βCH); 2.84 (dd, 1H, βCH); 3.94 (s, 2H, CH₂pNB); 4.05 (m, 1H, αCH); 7.10 (d, 1H, NH); 7.54 (d, 2H, aromatic); 8.13 (d, 2H, aromatic). ¹³C-NMR (150 MHz, CDCl₃) δ [ppm]: 26.1 (βCH₂); 27.1 (CH₂ pNB); 28.5 (CH₃ Boc); 53.5 (αCH); 81.1 (C Boc); 124.1 (CH aromatic); 129.9 (CH aromatic); 146.9 (2×C aromatic); 155.6 (C═O Boc); 175.4 (COOH).

### Example 4

### N^{α}-tert-butyloxycarbonyl-4-methylbenzyl-L-selenocysteine (Boc-L-Sec(MeBzl)-OH)

### (i) Synthesis of L-Selenocystine

41.8 g sodium borohydride (NaBH₄) (1103.8 mmol) in 200 mL cold H₂O was added drop-wise to 41.5 g (525.6 mmol) metallic selenium in 200 mL H₂O at 0° C. The flask was flushed continuously with nitrogen leaving the reaction vessel through two bleach traps in the fume hood. After the vigorous reaction subsided, the now grey metallic suspension was stirred for another 10 min at 0° C. before another equivalent of 41.5 g (525.6 mmol) of metallic selenium was added. The now reddish-brown mixture was stirred a further 15 min with gentle heating on a hot water bath to dissolve the rest of the selenium. 20 g of β-chloro-L-alanine (161.9 mmol) was dissolved in 250 mL H₂O (set to pH 9 with a few drops of 0.1 M NaOH) and added slowly into the reaction mixture over a period of two hours. After 16 h the solution was heated up on a hot water bath to 80° C. for 1 h, then cooled with a water-ice-bath and the pH was lowered with 6 N HCl to pH 2 (Caution: formation of toxic and irritating H₂Se gas). The solution was filtered through celite to remove metallic selenium and the precipate washed with 2 M HCl. 2.81 g (40.5 mmol) of hydroxylamine hydrochloride was added to reduce contaminating elemental selenium to H₂Se and the mixture was stirred another 2 h at room temperature. The yellow filtrate was flushed with N₂ for 30 min to remove any dissolved H₂Se gas and addition of solid NaOH pellets precipitated the product as a yellow solid at pH 6-6.5. The solution was kept in at 4° C. overnight and the precipitate was collected through vacuum filtration and dissolved again in a minimum of 2 M HCl. Insoluble impurities were filtered off and the pH of the filtrate was set to 6-6.5 again to precipitate the pure product. The precipitate was collected through vacuum filtration, washed with cold H₂O and ether, and finally dried over vacuum. 21.5 g of L-selenocystine was obtained (80% yield). ¹H-NMR, (600 MHz, D₂O/DCl), δ [ppm]: 4.21 (2H, dd, ³J=4.6 Hz, ³J=7.6 Hz, α-H), 3.32 (dd, 2H, ²J=14.1 Hz, ³J=4.6 Hz, β-H), 3.19 (dd, 2H, ²J=14.1 Hz, ³J=7.3 Hz, β-H) ¹³C (150 MHz, D₂O/DCl), δ [ppm]: 26.5 (CH₂), 52.7 (CH), 170.0 (COOH)

### (ii) Synthesis of Se-(4-Methylbenzyl)-l-Selenocysteine

15.9 g (419.1 mmol) of NaBH₄ was added in two portions to a solution of 20 g (59.9 mmol) of L-selenocystine, dissolved in 200 mL 1 M NaOH under argon atmosphere and at 0° C. The ice-water bath was removed after 15 min and the solution stirred until colorless. 100 mL of 2 M NaOH was added to the solution and 18.52 g (131.7 mmol) of α-chloro-p-xylene dissolved in 150 mL THF was added drop wise over 1 h at 0° C. The solution was stirred another 4 h at 0° C. before the product was precipitated as a white hydrochloride by addition of 6 M HCl. It was filtered, washed with cold ether and dried over night in vacuo over phosphorous pentoxide. 34 g of Se-(methylbenzyl)-l-selenocysteine hydrochloride was obtained (94% yield). ¹H-NMR, (600 MHz, d-DMSO), δ [ppm]: 8.61 (2H, bs, NH₂), 7.21 (2H, d, ³J=7.8 Hz), 7.09 (2H, d, ³J=7.8 Hz), 4.17 (1H, dd), 3.86 (2H, s), 2.95 (2H, dd), 2.25 (3H, s). ¹³C (150 MHz, CD₃OD+DCl+DSS), δ [ppm]: 170.71 (1C of COOH), 138.12 (1C aromatic), 136.81 (1C aromatic), 130.34 (2CH aromatic), 130.15 (2CH aromatic), 53.97 (1CH of NH—CH—COOH), 28.72 (1CH₂ of Se—CH₂-benzyl), 23.24 (1CH₂ of CH—CH₂—Se), 21.46 (3CH₃)

### (iii) Synthesis of N^{α}-tert-Butyloxycarbonyl-4-Methylbenzyl-L-Selenocysteine (Boc-Sec(MeBzl)-OH)

29 g (94.0 mmol) of the 4-methylbenzyl-l-selenocysteine hydrochloride was dissolved in 400 mL of water and 200 mL of dioxan. 26 g (187.9 mmol) of potassium carbonate was added to the solution, which was gently heated to solution. After cooling on a water-ice bath 24.61 g (112.8 mmol) di-tert-butyl-dicarbonate was dissolved in 200 mL of dioxan and added slowly to the reaction mixture. The solution was allowed to come to room temperature within 30 min and was stirred for another 2 h. Dioxan was removed in vacuo and 200 mL of 50% ether and 50% water was added to the remaining solution. The layers were separated and the aqueous layer washed a second time with ether. The aqueous solution was acidified with solid citric acid, extracted three times with ethyl acetate, washed with 10% citric acid and brine, dried over anhydrous MgSO₄, filtered and evaporated in vacuo. The product was recrystallized as a white solid in acetone/petrolether. 28.7 g of N^{α}-tert-butyloxycarbonyl-4-methylbenzyl-L-selenocysteine was obtained (82% yield). ¹H-NMR, (600 MHz, CDCl₃), δ [ppm]: 9.89 (1H, bs, COOH), 7.18 (2H, d, ³J=7.8 Hz), 7.09 (2H, d, ³J=7.8 Hz), 5.39 (1H, s, NH), 4.64 (1H, dd, ³J=6.6 Hz), 3.79 (2H, s), 2.95 (2H, dd, ³J=6.5 Hz), 2.32 (3H, s), 1.47 (9H, s) ¹H-NMR, (600 MHz, d-DMSO), δ [ppm]: 7.14 (2H, d), 7.07 (2H, d), 5.72 (1H, s, NH), 4.1 (1H, dd), 3.77 (2H, s), 2.77 (1H, dd), 2.68 (1H, dd), 2.24 (3H, s), 1.37 (9H, s); ¹³C (150 MHz, CDCl₃), δ [ppm]: 175.51 (1C of COOH), 155.43 (1C of O—CO—NH), 136.51 (2CH aromatic), 135.51 (2CH aromatic), 129.26 (1C aromatic), 128.78 (1C aromatic), 80.50 (1C of C(CH₃)₃), 53.26 (1CH of NH—CH—COOH), 28.29 (3CH₃), 27.57 (1CH₂ of Se—CH₂-benzyl), 25.46 (1CH₂ of CH—CH₂—Se), 21.07 (1CH₃)

On a 20 g scale an overall yield of 62% was achieved.

### Example 5

### 3-((4-methylbenzyl)selanyl)propanoic Acid (dSec(MeBzl)-OH)

### (i) Synthesis of Desamino-Selenocystine

Sodium borohydride (NaBH₄) (23.8 g, 628.3 mmol) in 200 mL cold H₂O was added drop-wise to metallic selenium (23.6 g, 299.2 mmol) in 80 mL H₂O at 0° C. The flask was flushed continuously with nitrogen leaving the reaction vessel through two bleach traps in the fume hood. After the vigorous reaction subsided, the now grey metallic suspension was stirred for another 10 min at 0° C. before another equivalent of metallic selenium (23.6 g, 299.2 mmol) was added. The now reddish-brown mixture was stirred a further 15 min with gentle heating on a hot water bath to dissolve the rest of the selenium. 3-Chloro-propanoic acid (10 g, 92.1 mmol) was dissolved in 120 mL H₂O (set to pH 9 with a few drops of 0.1 M NaOH) and added slowly into the reaction mixture over a period of 1 hour. After 16 h the solution was heated up on a hot water bath to 80° C. for 1 h, then cooled with a water-ice-bath and the pH was lowered with 6 M HCl to pH 2 (Caution: formation of toxic and irritating H₂Se gas). After the excess of Na₂Se₂ was destroyed, the solution was brought back to pH 9-10, the mixture filtered through celite to remove the excess of metallic selenium and the precipitate washed with 10% sodium bicarbonate. The aqueous solution was concentrated to half of its volume in vacuo, acidified with 6 M HCl to pH 2, extracted three times with ethyl acetate, washed with 10% citric acid and brine, dried over anhydrous MgSO₄, filtered and evaporated in vacuo. 4.2 g of desamino-selenocystine was obtained (30% yield). ¹H-NMR, (400 MHz, d-DMSO), δ [ppm]: 3.054 (2H, CH₂—COOH), 2.680-2.727 (2H, CH₂—Se).

### (ii) Synthesis of 3-((4-methylbenzyl)selanyl)propanoic Acid (dSec(MeBzl)-OH)

NaBH₄ (3.7 g, 96.7 mmol) was added in small portions to a solution of L-selenocysteine (4.2 g, 13.8 mmol), dissolved in 100 mL 1 M NaOH under argon atmosphere and at 0° C. The ice-water bath was removed after 15 min and the solution stirred until colorless. α-chloro-p-xylene (4.27 g, 30.4 mmol) was added drop wise to the reaction mixture at 0° C. The reaction mixture was let to warm up to room temperature and stirred for another 4 h. The solution was washed twice with ether, acidified with HCl, extracted three times with ethyl acetate, dried over anhydrous MgSO₄, filtered and evaporated in vacuo. The product was recrystallized as a white solid in acetone/petrolether, yielding 4.1 g of 3-((4-methylbenzyl)selanyl)propanoic acid (41% yield, >98% purity by HPLC). ¹H-NMR, (400 MHz, d-DMSO), δ [ppm]: 7.169-7.189 (2H, d, ³J=8 Hz), 7.083-7.103 (2H, d, ³J=8 Hz), 3.789 (2H, s), 2.680-2.718 (2H, dd, ³J_{A}=6.4 Hz ³J_{B}=6.8 Hz, CH₂—COOH), 2.600-2.638 (2H, dd, ³J_{A}=6.4 Hz ³J_{B}=6.8 Hz, Se—CH₂), 2.25 (3H, s). ¹³C (150 MHz, d-DMSO), δ [ppm]: 173.72 (1C of COOH), 136.96 (1C aromatic), 135.98 (1C aromatic), 129.35 (2CH aromatic), 129.10 (2CH aromatic), 35.88 (1CH₂ of Se—CH₂-benzyl), 35.52 (1CH₂ of CH₂—CH₂—COOH), 26.46 (1CH₂ of Se—CH₂—CH₂), 21.12 (1CH₃).

### Example 6

All reactions involving tellurium were conducted in a three-necked flask fitted with a reflux condenser and under a continuous flow of argon, with the exhaust vented through a NaOCl trap.

### Tellurocystine (Tec₂)

To a stirred suspension of elemental tellurium powder (12.92 g, 101.25 mmol) in H₂O (75 mL) was added a solution of NaBH₄ (15.32 g, 405 mmol) in H₂O (75 mL). An ice bath was applied periodically as the reaction became exothermic. Stirring was continued at 50° C. until the solution became clear and colourless, which was then heated to reflux to destroy the excess NaBH₄. An additional 12.92 g (101.25 mmol) of tellurium powder was added and heating was continued until the tellurium was completely dissolved to give a dark purple solution of Na₂Te₂. After being allowed to cool to room temperature, a solution of β-chloroalanine (5 g, 40.5 mmol) in degassed H₂O (60 mL, pH adjusted to 9 with NaOH) was added dropwise over 1 h. After stirring for 16 h in the dark, the reaction was placed in an ice bath and concentrated HCl was added dropwise to reduce the pH to 0-1, forming a black precipitate and H₂Te gas. The mixture was allowed to warm to room temperature with continued stirring for 1 h, filtered over Celite and the volume of solvent was reduced in vacuo to 70-80 mL. The pH of the solution was adjusted to 6.5 with NaOH and kept at 4° C. for 1 h. The resulting precipitate was filtered, washed with H₂O and dried in vacuo to give tellurocystine as a dark yellow powder (6.375 g, 14.78 mmol, 73%). MS [M+H] m/z: calc 436.9, found 436.8. ¹H-NMR, (400 MHz, D₂O/DCl), δ [ppm]: 4.12 (1H, t, J=6.4 Hz, Hα); 3.53 (1H, dd, J=12.8, 6.4 Hz, Hβ); 3.50 (1H, dd, J=13.2, 6.8 Hz, Hβ). ¹³C (100 MHz, D₂O/DCl), δ [ppm]: 170.6 (CO), 54.9 (CH), 0.0 (CH₂).

### Te-(4-methylbenzyl)tellurocysteine

To a solution of tellurocystine (1 g, 2.32 mmol) in 1 M NaOH (5 mL) at 0° C. was added a solution of NaBH₄ (702 mg, 18.56 mmol) in H₂O (5 mL). The pH of the solution was adjusted to 11 with 6 M HCl then allowed to warm to room temperature. The resulting clear colourless solution was cooled again to 0° C. and a solution of 4-methylbenzyl chloride (783 mg, 5.56 mmol) in THF (5 mL) was added dropwise over 10 min. The solution was stirred at 0° C. for 30 min in the dark before the addition of 6 M HCl to lower the pH to 1, giving a white precipitate. The precipitate was filtered and washed with H₂O then Et₂O (under argon), then dried in vacuo to give Te-(4-methylbenzyl)tellurocysteine hydrochloride as an off-white powder (1.42 g, 85%). MS [M+H] m/z: calc 324.0, found 324.1. ¹H-NMR, (400 MHz, MeCN-d₃/D₂O/DCl), δ [ppm]: 7.17 (2H, d, J=8 Hz, Ar—H); 7.08 (2H, d, J=8 Hz, Ar—H); 4.19 (1H, t, J=6.4 Hz, Hα); 4.04 (2H, q, J=2 Hz, 7.2 Hz, Ar—CH₂); 3.09 (1H, dd, J=13.6, 6.4 Hz, Hβ), 2.94 (1H dd, J=13.6, 6.4 Hz, Hβ); 2.25 (3H, s, CH₃). ¹³C-NMR, (400 MHz, MeCN-d₃/D₂O/DCl), δ [ppm]: 170.0 (CO), 138.1, 136.1, 129.5, 128.5, 54.1 (CH), 20.4, 7.4, 1.3 (CH₂).

### Comparative Example 1

### Sec(Acm)[1]-OT(SEQ ID NO:27) and Sec(Acm)[1,6]-OT (SEQ ID NO:28)

SEQ ID NO: 27
Sec(Acm)YIQNCPLG-NH₂
SEQ ID NO: 28
Sec(Acm)YIQNSec(Acm)PLG-NH₂

The peptides were assembled on a 0.25 mmol scale via standard Boc-SPPS using HBTU-mediated in situ protocol (Schnölzer, et al., Int. J. Pept. Protein Res., 1992, 40:180-193) on a MBHA resin (0.6 mmol/g) with no special adjustments for the coupling of Boc-L-Sec(Acm)-OH. Standard HF cleavage of 300 mg dry resin yielded 105 mg crude Sec(Acm)[1,6]-OT (87% yield) and 60 mg crude Sec(Acm)[1]-OT (63% yield), which were purified by preparative RP-HPLC resulting in 14 mg pure Sec(Acm)[1,6]-OT (12% overall yield) and 22 mg Sec(Acm)[1]-OT (22% overall yield).

### Comparative Example 2

### [C1U]-OT (SEQ ID NO:29)

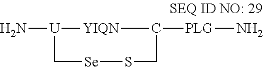

Sec(Acm)[1]-OT (12 mg) was dissolved in acetic acid (AcOH, 60 mL). 2N HCl (2.4 mL) was added just before addition of 0.1 M I₂/MeOH (2.7 mL). The reaction was monitored by MS, analytical RP-HPLC and LCMS. After 20 min the reaction was complete, quenched with 2M ascorbic acid until it turned colorless and was directly purified by preparative RP-HPLC yielding [C1U]-OT (4.8 mg, 40% yield).

### Comparative Example 3

### [C1,6]-OT (SEQ ID NO:30)

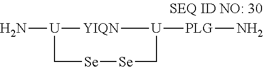

Sec(Acm)[1,6]-OT (3.4 mg) was dissolved in AcOH (15 mL). 2 N HCl (680 mL) was added just before the addition of 0.1 M I₂/MeOH (2.7 mL). The reaction was monitored by MS, analytical RP-HPLC and LCMS. After 20 min the reaction was complete, quenched with 2 M ascorbic acid until it turned colorless and was directly purified by semi-preparative RP-HPLC yielding [C1,6U]-OT (2 mg, 59% yield).

### Comparative Example 4

### Sec(pNB)[6]-OT (SEQ ID NO:31)

SEQ ID NO: 31
CYIQNSec(pNB)PLG-NH₂

The peptide was assembled on a 0.25 mmol scale via standard Boc-SPPS using HBTU-mediated in situ protocol (Schnölzer et al., ibid) on a MBHA resin (0.6 mmol/g) with no special adjustments for the coupling of Boc-L-Sec(pNB)-OH. Standard HF cleavage of 300 mg dry resin yielded 65 mg crude Sec(pNB)[6]-OT (66% yield), which was purified by preparative RP-HPLC resulting in 46 mg pure Sec(pNB)[6]-OT (47% overall yield).

### Comparative Example 5

### Sec(pNB)[6]-OT (SEQ ID NO:32)

SEQ ID NO: 32
CYIQNSec(pNA)PLG-NH₂

7.5 mg of Sec(pNB)[6]-OT were dissolved in 4 mL 80% AcOH and 40 mg (50 eq) nano-zinc were added at 0° C. for 1 h. The nano-zinc particles were centrifuged down and purification by semi-preparative RP-HPLC yielded 3 mg pure Sec(pNA)[6]-OT (40% yield).

### Comparative Example 6

### [C6U]-OT (SEQ ID NO:33)

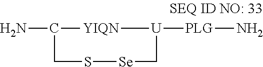

3 mg of Sec(pNA[6]-OT were dissolved in 17 mL of AcOH. 600 mL of 2 N HCl were added just before the addition of 1.3 mL 0.1 M I₂/MeOH and the solution was stirred at 25° C. The reaction was monitored by MS, analytical RP-HPLC and LCMS. After 20 min the reaction was complete and quenched with 2 M ascorbic acid until it turned colorless. Purification by semi-preparative RP-HPLC yielded 0.5 mg pure [C6U]-OT (17% yield).

### Example 7

### [C1,6U]-OT-OH (SEQ ID NO:3)

The peptide was assembled via Boc-chemistry SPPS on a PAM resin using the [2-(1H-benzotriazol-1-yl)-1,1,3,3-tetramethyl uronium hexa-fluorophosphate] (HBTU)—mediated in situ neutralization protocol (Schnolzer et al. ibid). The selenocysteine building block used in the peptide synthesis was Boc-L-Sec (MeBzl)-OH as prepared in Example 4. HF deprotection or cleavage was performed by treatment of the dried resin (300 mg) with 10 mL HF/p-cresol (9.1 v/v) for 2 h at 0° C. Following evaporation of the HF, the peptide was precipitated and washed with cold ether, filtered and redissolved directly in 0.1 M NH₄HCO₃ (pH 8.4, c=0.1 μM) for direct oxidation. Oxidation was monitored by RP-HPLC, LC-MS and MS, and the peptide was purified using preparation C₁₈ RP-HPLC.

### Comparative Example 7

### [Te—Te]-Oxytocin, [Cys1,6Tec]-Oxytocin

The linear [Cys1,6 β-chloroalanine]-Oxytocin precursor was assembled using standard Boc-SPPS with HBTU/DIEA amino acid activation on MBHA-polystyrene resin (0.6 mmol/g). The following side-chain protecting groups were used: Asn(Xan), Gln(Xan), Tyr(2-BrZ). Boc-Ser-OH was coupled without side chain protection and subsequently converted to β-chloroalanine (1:1 Ph₃P:Cl₃CN in DCM, 16 h, 10 eq to resin loading) before continuing peptide assembly.

To a stirred suspension of elemental tellurium powder (153 mg, 1.2 mmol) in H₂O (1 mL) was added a solution of NaBH₄ (182 mg, 4.8 mmol) in H₂O (1 mL) and heated at 50° C. for 30 min to give a clear colourless solution, which was then heated to reflux to destroy the excess NaBH₄. An additional 153 mg (1.2 mmol) of tellurium powder was added and heating was continued until all the tellurium was completely dissolved to give a dark purple solution of Na₂Te₂, which was diluted with 12 mL of degassed DMF and cooled to 0° C. Resin-bound [Cys1,6 β-chloroalanine]-Oxytocin (133 mg, pre-swollen in 1 mL DMF) was added to the solution, which was allowed to warm to room temperature and stirred for 2 h. TFA was added to acidify the mixture to pH 0-1 producing a black precipitate and H₂Te gas. Stirring was continued for 20 min, the resin was filtered off and washed with DMF/H₂O then DMF before removal of the final N-terminal Boc group. The resin was then washed with DMF, DCM/MeOH and dried under nitrogen.

113 mg of the dried resin was treated with 5 mL HF/p-cresol (9:1, v/v) at 0° C. for 45 min and the product was precipitated and washed with cold Et₂O. The precipitate was dissolved and lyophilized from 50% acetonitrile/0.05% TFA/H₂O to give [Cys1,6Tec]-OT as a pale yellow powder (26 mg, 64% from [Cys1,6 β-chloroalanine]-OT resin, based on resin loading).

Crude [Cys1,6Tec]-OT (26 mg) was dissolved in 5% acetonitrile/0.05% TFA/H₂O (10 mL) and purified on a Vydac 28TP510 column running at a flow rate of 6 mL/min with a linear gradient of 10 to 40% B in 30 min. Fractions containing the correct product were combined and lyophilized to give [Cys1,6Tec]-OT (pale yellow powder, 1.5 mg). HRMS [M+H] m/z: calc 1203.3126, found 1203.2224.

### Example 8

### Stability Assay

300 μL rat plasma and human serum (Sigma Aldrich) was incubated at 37° C. for 30 min. 50 mL of peptide sample (0.3 mM) in 0.1 M phosphate buffer, pH 7.2 was added to the rat plasma and human serum. The vortexed mixture was incubated at 37° C. Aliquots (30 μL) were taken at 1, 2, 3, 4, 12, 24 and 48 h, quenched with extraction buffer (70 μL) consisting of 50% ACN/H₂O, 0.1 M NaCl, and 1% TFA, chilled on ice for 5 min, centrifuged (14000 rpm, 10 min) and analyzed (2×20 mL injection) by RP-HPLC and LCMS. n=3-6.

The stability of peptides of SEQ ID NOs:1 (OT), 2 (AVP) and SEQ ID NO:11 [C1U]-OT and SEQ ID NO:3 [C1,6U]-OT-OH was assessed in rat plasma and human serum (Table 2). The stability of peptides incubated in rat plasma correlated well with stability in human serum. The positive control was OT and AVP incubated in phosphate buffer at pH 7.4 and 37° C., where no degradation was observed over a period of 48 h. AVP showed a very short half-life of 2 h compared to OT with 12 h, and was not detectable by analytical RP-HPLC and LCMS after 4 h. The selenium analogues [C1U]-OT and [C1,6U]-OT-OH exhibited an increase in stability in relation to OT.

TABLE 2
Metabolic Half Life

| Peptide | Half life (h) |
|---|---|
| Oxytocin | 11.6 ± 1.9 |
| Vasopressin | 1.7 ± 0.6 |
| [C1U]-OT | 18.6 ± 3.0 |
| [C1,6U]-OT-OH | 25.0 ± 6.7 |

### Example 9

### Human OTR, V1aR, V1bR

OT, V₁ₐ and V_{1b} cDNA were obtained from Origene technologies inc (Rockville, USA). Dulbecco's modified Eagle's medium, Lipofectamine2000 and Fluo-4-AM were purchased from Invitrogen (Mulgrave, VIC, Australia) and the Wheat Germ Agglutinin (WGA) PVT SPA Scintillation Beads was from GE Healthcare Bio-Sciences (Ryadelmere, NSW, Australia). Complete protease inhibitor cocktail and FuGENE® 6 Transfection Reagent were from Roche Diagnostics (Castle Hill, NSW, Australia). [Tyrosyl-2,6-³H]-oxytocin; 46.3 Ci/mmol, ¹²⁵I-linear vasopressin V₁ₐ receptor antagonist ([¹²⁵I]phenylacetyl-D-Tyr(Me)-Phe-Gln-Asn-Arg-Pro-Arg-Tyr-NH₂); 2200 Ci/mmol, ³H-Vasopressin, 8-arginine, [Phenylalanyl-3,4,5-3H(N,)]; 61.2Ci/mmol, FlashBLUE GPCR scintillating beads, TopSeal-A 96-well sealing film and 384-well white optiplates were from PerkinElmer Life Sciences (Knoxfield, VIC, Australia). Costar 96-well white plates with clear bottom were obtained from Corning (Lindfield, NSW, Australia). The IP-one HTRF assay kit was from CisBio International (30204 Bagnols-sur-Cèze, France).

### Transfection and Membrane Preparation

COS-1 cells grown in Dulbecco's modified Eagle's medium and 5% fetal bovine serum in 150 mm plates were transiently transfected with plasmid DNA (25 μg) encoding OTR, V₁ₐR or V_{1b}R using Lipofectamine2000 reagent (50 μl). The cells were harvested 48 h post transfection and homogenized using an Ultra turrax homogeniser (22000/min) in OT buffer for OTR (50 mM Tris-HCl, 5 mM MgCl₂, pH 7.4) or Vasopressin buffer for V₁ₐR and V_{1b}R (50 mM Tris-HCl, 10 mM MgCl₂, pH 7.4) with complete protease inhibitor cocktail. The homogenate was centrifuged at 2000 rpm for 10 min and the resulting supernatant was centrifuged at 14000 rpm for 30 min. The pellet was resuspended in appropriate buffer without protease inhibitor containing 10% glycerol and stored at −80° C. until assayed.

### Radioligand Binding Assay

Receptor binding assays were performed using FlashBLUE GPCR scintillation beads (OTR and V₁ₐR) or Wheat Germ Agglutinin (WGA) PVT SPA Scintillation Beads (V_{1b}R). Reactions containing increasing concentrations of competing OT analogue (10 pM to 10 μM), SPA beads (100 mg of FlashBLUE beads, 200 mg of WGA beads), OTR/V₁ₐR/V_{1b}R membrane preparation (5 μg protein) and radioligand (³H-OT for OTR (2 nM), ¹²⁵I-V₁ₐ linear antagonist for V₁ₐR (21 pM), ³H-AVP for V_{1b}R (0.5 nM) in assay buffer with 0.1% BSA were established in 96-well white polystyrene plates with clear flat bottoms. The assays were performed in triplicate, in a total reaction volume of 80 μL. The plates were sealed with TopSeal-A film and incubated with shaking for 1 h at room temperature. Radioligand binding was detected using a Wallac 1450 MicroBeta scintillation counter (PerkinElmer Life Sciences).

### Transfection of Cells for Functional Assays

COS-1 cells were transiently transfected with plasmid DNA encoding the OT/V₁ₐN_{1b} receptor using 14.5 μg DNA/29 μL Lipofectamine2000 or 18 μg DNA/54 μL Fugene in Dulbecco's modified Eagle's medium.

### Functional IP-One Homogenous Time-Resolved Fluorescence (HTRF) Assay

Assays measuring IP₁-accumulation were performed 48 h after transfection according to manufacturer's protocol. Briefly, cells were incubated with increasing concentration of OT analogue in stimulation buffer (Hepes 10 mM, CaCl₂ 1 mM, MgCl₂ 0.5 mM, KCl 4.2 mM, NaCl 146 mM, glucose 5.5 mM, LiCl 50 mM pH 7.4) for 1 h in 37° C., 5% CO₂ in white 384-well optiplates. Cells were lysed by the addition of the HTRF reagents (the europium cryptate-labeled anti-IP₁ antibody and the d2-labeled IP₁ analogue) diluted in lysis buffer. The assays were incubated for 1 h at room temperature. The emission signals at 590 nm and 665 nm were measured after excitation at 340 nm using the Envision multilabel plate reader (PerkinElmer Life Sciences).

### Functional Analysis Using the Fluorescent Imaging Plate Reader (FLIPR)

COS-1 cells transfected with OTR, V₁ₐR or V_{1b}R were plated 24 h prior to the experiment at a density of 35,000-50,000 cells/well on black-walled 96-well imaging plates (Corning, Lowell, Mass., USA). Cells were loaded for 30 min at 37° C. with Fluo-4-AM (4.8 μM) in physiological salt solution (PSS; composition NaCl 140 mM, glucose 11.5 mM, KCl 5.9 mM, MgCl₂ 1.4 mM, NaH₂PO₄ 1.2 mM, NaHCO₃ 5 mM, CaCl₂ 1.8 mM, HEPES 10 mM) containing 0.3% fatty-acid-free BSA. To allow for complete de-esterification of the dye, cells were washed 5-10 min with PSS before transfer to a FLIPR^{TETRA} (Molecular Devices, Sunnyvale, Calif.) fluorescent plate reader. Ca²⁺ responses were measured using a cooled CCD camera with excitation at 470-495 nM and emission at 515-575 nM. The baseline fluorescence was set to a minimum of 1000 arbitrary fluorescence units by adjusting camera gain and excitation intensity. Compounds were added as 3× concentrated stock solutions in PSS, with 10 baseline fluorescence readings prior to compound addition followed by fluorescence reading every second for 180 s.

### Data Analysis

As previously described, raw fluorescence data was converted to ΔF/F values by subtracting baseline fluorescence readings from subsequent time points and dividing by baseline fluorescence values. For concentration-response curves, maximum ΔF/F values after addition of compounds were plotted against agonist concentration and normalized to the response elicited by oxytocin. A 4-parameter Hill equation with Hill coefficient=1 was fitted to the data using GraphPad Prism (Version 4.00, San Diego, Calif.).

### Human V2R

### Materials

The cDNA plasmid clones for human V2 receptor were obtained as a gift from Ralf Schülein (FMP, Berlin). The V2 receptor sequence was inserted into the pEGFP-N1 plasmid (Clontech, Saint-Germain-en-Laye, France) using SacI and HindIII restriction sites (to yield a C-terminal GFP fusion protein) or into pKaede-MN1 (MBL, Japan) using EcoRI and HindIII restriction sites (to yield the wild type receptor). OT and AVP were obtained from Sigma Aldrich (Vienna, Austria). [Phenylalanyl-3,4,5-3H(N)]-8-arginine vasopressin; 61.2 Ci/mmol was from PerkinElmer Life Sciences (Boston, Mass.). Complete protease inhibitor cocktail was from Roche Diagnostics (Mannheim, Germany). In addition, the following materials were used: DMEM high glucose medium, with L-glutamine and gentamicin, an antibiotic added to DMEM (PAA, Pasching, Austria), geneticin G418-BC liquid—50 mg/mL (Biochrom, Berlin, Germany), [2,8-3H]-adenine; 27.2 Ci/mmol (PerkinElmer Life Sciences, Boston, Mass.), RO201724—a cell-permeable, selective inhibitor of cAMP-specific phosphodiesterase (Calbiochem, Merck, Darmstadt, Germany), forskolin—a cell-permeable diterpenoid that possesses anti-hypertensive, positive inotropic, and adenylyl cyclase activating properties (Sigma-Aldrich, Vienna, Austria) and glass microfiber binder free membranes Whatman GF/A 1.6 μm (Whatman International Ltd, Kent, UK). All other Chemicals were purchased from Sigma-Aldrich (Vienna, Austria).

### Cell Culture and Cellular Transfections and Membrane Preparation

The conditions for the propagation of HEK293 cells, creation of stably transfected V2R cell lines and for transient transfections were as described previously (Klinger et al. 2002, Naunyn Schmiedebergs Arch. Pharmacol., 366:287-298). Cells were harvested and membranes were prepared as described previously (Klinger et al. 2002, ibid). For binding assays on intact and lysed cells, the confluent monolayer was rinsed three times with phosphate-buffered saline (PBS) and subsequently detached by incubating the cells for 10 min at 37° C. in PBS containing EDTA; cells were rinsed off without any additional mechanical manipulation, harvested by centrifugation (5 min at 500 g) and resuspended in DMEM. To obtain cell lysis, an aliquot of the harvested cells was subjected to two freeze-thaw cycles (using liquid nitrogen) followed by brief sonication in a small volume of PBS (˜0.3-0.5 mL); the suspension was subsequently diluted with DMEM to match the suspension of intact cells.

### Radioligand Binding Assay

Membranes (25-100 μg/assay) from HEK293 cells stably expressing the V2 receptors were incubated in a final volume of 200 μL containing 50 mM Tris-HCl, 5 mM MgCl₂, 0.1% BSA, pH 7.8 and logarithmically spaced concentrations (0.5-25 nM) of [³H]-AVP (agonist). Competitive replacement of native ligand by synthetic peptide analogues or control peptides was performed in the presence of various concentrations of competing peptide (0.3 nM-10 μM). After 60 min at room temperature, the reaction was terminated by rapid filtration over glass fiber filters. Nonspecific binding was determined in the presence of 1 μM AVP. Specific binding represents the difference between total and binding. Incubations were considered to represent binding to intact cells only if >90% of the cells became adherent upon replating after a mock incubation.

Functional cAMP Accumulation Assay

Cells were grown in 6-well plates. The adenine nucleotide pool was metabolically labeled by incubating confluent monolayers for 16 h with [³H]adenine (1 μCi/well) as described (Klinger et al 2002, ibid). After the preincubation, fresh medium was added that contained 100 μM RO201724 (a phosphodiesterase inhibitor). After 4 h, cAMP formation was stimulated by the V2R agonist AVP (0.05 nM-1 μM), oxytocin (0.06 nM-1 μM), synthetic oxytocin analogues (0.7 nM-10 μM) or forskolin (30 μM). Assays were performed at least three times (n=3) in triplicate. The formation of [³H]cAMP was determined according to Klinger et al. (Klinger et al 2002, ibid).

The peptides were tested for activity at the human OTR, V₁ₐR, V_{1b}R and V₂R (Table 3). Binding data were obtained in radioligand binding assays measuring the displacement of ³H-OT on the hOTR, of ¹²⁵I-V₁ₐ linear antagonist on the hV₁ₐR, and of ³H-AVP on the hV_{1b}R and hV₂R. The hOTR, V₁ₐR and V_{1b}R were expressed in COS-1 cells and the V₂R in HEK293 cells. The functional ability of the analogues to signal through the individual receptors were investigated with a HTRF (homogeneous time-resolved fluorescence) assay measuring the generation of IP-one for the hOTR, hV₁ₐR and hV_{1b}R, with a FLIPR (fluorescent imaging plate reader) to quantify calcium signalling for the hOTR, hV₁ₐR and hV_{1b}R, and with a cAMP assay measuring generation of cAMP for the V₂R

TABLE 3
Binding affinity data for OT and AVP analogues at the human OT, V₁ₐ, V_{1b} and
V₂ receptors. The IC₅₀ values [nM] were obtained from radioligand binding assays.

| | | hOTR | hV1aR | hV1bR | hV2R |
|---|---|---|---|---|---|
| SEQ ID# | | IC50[nM] | IC50 [nM] | IC50 [nM] | IC50 [nM] |
| | Control Compounds | | | | |
| 1 | OT | 1.5 ± 0.37 | 5.1 ± 0.91 | 2323 ± 1535 | 1780 ± 235 |
| 34 | dOT | 1.7 | n.d. | 351 ± 125 | 488 ± 116 |
| 2 | AVP | 30 ± 6.8 | 0.38 ± 0.07 | 0.67 ± 0.35 | 1.15 ± 0.61 |
| 35 | dAVP | 104 | n.d. | 0.64 ± 0.30 | 2.05 ± 1.14 |
| | OT Analogues | | | | |
| 36 | [CH₂—S]-OT | 3.1 ± 0.59 | 9.8 ± 2.3 | n.d. | >105 |
| 29 | [Se—S]-OT | 0.58 ± 0.04 | 3.18 ± 0.77 | n.d. | 2259 |
| 33 | [S—Se]-OT | 1.45 ± 0.63 | 8.2 ± 1.6 | n.d. | 5974 ± 2888 |
| 30 | [Se—Se]-OT | 538 ± 64 | 127 ± 29 | 1756 ± 188 | 3349 ± 804 |
| 37 | [Te—Te]-OT | 8 | n.d. | n.d. | n.d. |
| 38 | d[Se—Se]-OT | 1.9 | n.d. | 283 ± 123 | n.d. |
| 39 | [S—S]-OT-OH | 5454 ± 1633 | 492 ± 118 | 6045 ± 2149 | >105 |
| 3 | [Se—Se]-OT-OH | 1276 ± 451 | 114 ± 28 | >105 | 4407 ± 2218 |
| 4 | d[Se—Se]-OT-OH | 155 | n.d. | 1016 ± 697 | n.d. |

n.d. . . . not determined
n = 3-6 except for single values where n = 1

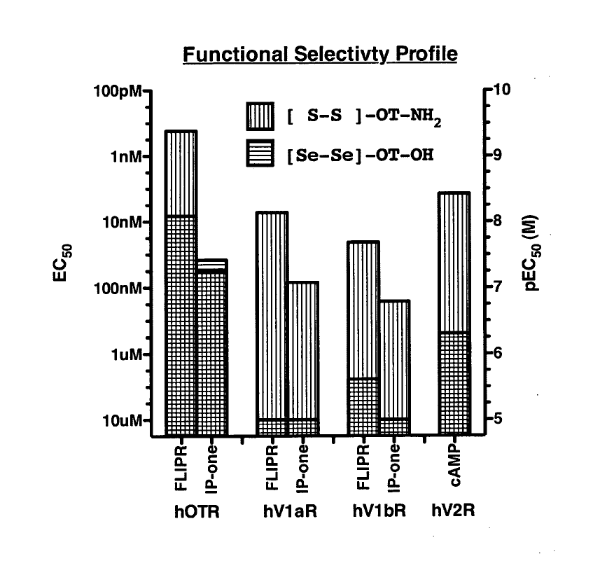

As shown in Tables 3-6, SEQ ID NO:29 [C1U]-OT, SEQ ID NO:33 [C6U]-OT and [CH₂—S]-OT SEQ ID NO:36 retained binding affinity and functional efficacy at the hOTR and hV₁ₐR. SEQ ID NO:37 [Te—Te]-OT also preserved binding affinity and functional efficacy for the hOTR, but lost its ability to signal on the hV₁ₐR. The affinity for SEQ ID NO:30 [C1,6U]-OT dropped 500-fold for the hOTR (538 nM) and 25-fold for the hV₁ₐR (127 nM) compared to OT, but it retained its ability to signal through the hOTR (1.4 nM), hV₁ₐR (35.9 nM) and hV_{1b}R (7.75 nM). SEQ ID NO:38 d[C1,6U]-OT preserved its full binding affinity for the hOTR and showed a 10-fold enhancement in binding on the hV_{1b}R (283 nM) compared to oxytocin (2323 nM). 3-30 fold drops in functional efficacies were observed at the hOTR, hV_{1b}R and V₂R, and a 200-fold drop on the hV₁ₐR. SEQ ID NO:39 OT-OH lost most of its binding ability on all four receptors. Its functional efficacy dropped 10-30 fold on the hOTR (251 nM), V_{1b}R (1780 nM) and V₂R (100 nM), while its ability to stimulate IP-one signalling through the V₁ₐR (148 nM) stayed similar to that of OT (83 nM). SEQ ID NO:3 [C1,6U]-OT-OH exhibited a 1000-fold drop in binding on the hOTR (1276 nM) compared to OT (1.5 nM) and turned into partial agonist (Eₘₐₓ=40-60%) on the hOTR with a similar EC₅₀ to OT. SEQ ID NO:3 [C1,6U]-OT-OH lost most of its ability to signal through the vasopressin receptors with no activity observed on hV₁ₐR and hV_{1b}R at concentration up to 10 μM, and an EC₅₀ of 495 nM for the V₂R. [C1,6U]-OT-OH displayed a 14000-fold (FLIPR assay) and a 1200-fold (IP-one assay) selectivity improvement for the hOTR over the hV₁ₐR, a 120-fold (FLIPR assay) and a 600-fold (IP-one assay) selectivity improvement over the hV_{1b}R, and a 130-fold selectivity improvement over the V₂R compared to the endogenous ligand oxytocin. SEQ ID NO:4 d[C1,6U]-OT-OH showed a 10-fold loss on binding affinity to the hOTR and acted as a partial agonist (Eₘₐₓ=50-70%) on the hOTR with an EC₅₀ similar to that of OT. No functional activity was observed on the V₁ₐR up to a concentration of 100 μM and functional efficacy also dropped slightly for the V_{1b}R and V₂R. The functional selectivity profile is shown in FIG. 1.

TABLE 4
Functional potencies for OT and AVP analogues at the human OT, V₁ₐ, V_{1b} and V₂ receptors.
The EC₅₀ values [nM] and the maximal efficiency Eₘₐₓ [%] (normalized to OT for the hOTR
and to AVP for the AVPRs) were obtained from a functional IP-One assay and FLIPR assay for
the hOTR, hV₁ₐR and hV_{1b}R, and from a functional cAMP assay on the hV₂R.
Functional IP-One Assay

| | | hOTR | | hV1aR | | hV1bR | |
|---|---|---|---|---|---|---|---|
| SEQ | | EC50 | Emax | EC50 | Emax | EC50 | Emax |
| ID# | | [nM] | [%] | [nM] | [%] | [nM] | [%] |
| | Control Compounds | | | | | | |
| 1 | OT | 11.5 ± 2.4 | 100 | 83 ± 29 | 79 ± 9 | 161 ± 139 | 105 ± 16 |
| 34 | dOT | 0.71 | 100 | n.d. | n.d. | 470 ± 422 | 67 ± 10 |
| 2 | AVP | 55 ± 12 | 80 ± 10 | 0.58 ± 0.18 | 100 | 0.3 ± 0.06 | 100 |
| 35 | dAVP | 24 | 47 | n.d. | n.d. | 0.28 ± 0.08 | 70 ± 3 |
| | OT Analogues | | | | | | |
| 36 | [CH₂—S]-OT | 32 ± 9.4 | 64 ± 14 | 263 ± 18 | 75 ± 8 | n.d. | n.d. |
| 29 | [Se—S]-OT | 2.57 ± 1.0 | 84 ± 6 | 13 ± 0 | 96 ± 19 | n.d. | n.d. |
| 33 | [S—Se]-OT | 221 ± 172 | 55 ± 9 | 91 ± 32 | 67 ± 20 | n.d. | n.d. |
| 30 | [Se—Se]-OT | 18 ± 12 | 94 ± 18 | 269 ± 99 | 70 ± 13 | 510 ± 172 | 103 ± 12 |
| 37 | [Te—Te]-OT | 13.3 ± 11 | 98 | >10⁴ | — | n.d. | n.d. |
| 38 | d[Se—Se]-OT | 400 | 72 | n.d. | n.d. | 538 ± 27 | 96 ± 22 |
| 39 | [S—S]-OT-OH | 251 ± 94 | 82 ± 13 | 148 ± 59 | 77 ± 24 | 1709 ± 979 | 96 ± 6 |
| 3 | [Se—Se]-OT-OH | 38 ± 7.4 | 60 ± 4 | >10⁵ | — | >10⁵ | — |
| 4 | d[Se—Se]-OT-OH | 0.61 | 71 | n.d. | n.d. | 471 ± 422 | 63 ± 4 |

n.d. not determined
n = 3-6 except for single values where n = 1

TABLE 5
Functional FLIPR Assay
Functional FLIPR Assay

| | | hOTR | | hV1aR | | hV1bR | |
|---|---|---|---|---|---|---|---|
| SEQ | | EC₅₀ | Eₘₐₓ | EC₅₀ | Eₘₐₓ | EC₅₀ | Eₘₐₓ |
| ID# | | [nM] | [%] | [nM] | [%] | [nM] | [%] |
| | Control Compounds | | | | | | |
| 1 | OT | 0.41 | 100 | 7.27 | 71 | 19.8 ± 4.5 | 107 |
| 34 | dOT | 0.39 | 89 | 46.1 | 54 | 15.88 ± 5.96 | 100 |
| 2 | AVP | 1.1 | 78 | 0.50 | 100 | 0.14 ± 0.09 | 100 |
| 35 | dAVP | 1.65 | 83 | 0.30 | 90 | 0.23 ± 0.20 | 117 |
| | OT Analogues | | | | | | |
| 36 | [CH₂—S]-OT | n.d. | n.d. | n.d. | n.d. | n.d. | n.d. |
| 29 | [Se—S]-OT | 0.49 | 85 | 11.9 | 90 | 11 | 82 |
| 33 | [S—Se]-OT | 3.15 | 82 | 240 | 104 | 115 | 73 |
| 30 | [Se—Se]-OT | 1.39 | 86 | 35.9 | 100 | 7.75 ± 3.2 | 82 |
| 37 | [Te—Te]-OT | 11.3 ± 7.5 | 86 | 4277 ± 3378 | — | 100.7 ± 37.6 | 104 |
| 38 | d[Se—Se]-OT | 4.9 | 89 | 1443 | 100 | 68.6 | 92 |
| 39 | [S—S]-OT-OH | n.d. | n.d. | n.d. | n.d. | 1754 | 107 |
| 3 | [Se—Se]-OT-OH | 8.19 | 38 | >10⁵ | — | 2461 ± 353 | 63 |
| 4 | d[Se—Se]-OT-OH | 28.5 | 49 | >10⁵ | — | 279 | 58 |

n.d. not determined
n = 2-3, except for single values, where n = 1

TABLE 6
Functional cAMP Assay
Functional cAMP Assay

| SEQ | | hV2R | Eₘₐₓ |
|---|---|---|---|
| ID# | | EC₅₀ [nM] | [%] |
| | Control | | |
| | Compounds | | |
| 1 | OT | 3.78 ± 4.70 | 98 ± 23 |
| 34 | dOT | 1.16 ± 1.18 | 85 ± 13 |
| 2 | AVP | 0.12 ± 0.08 | 100 |
| 35 | dAVP | 0.14 ± 0.16 | 108 ± 7 |
| | OT Analogues | | |
| 38 | d[Se—Se]-OT | 11.5 ± 1.6 | 134 ± 17 |
| 39 | [S—S]-OT-OH | 99.5 ± 31 | 124 ± 30 |
| 3 | [Se—Se]-OT-OH | 495 ± 90 | 114 ± 46 |
| 4 | d[Se—Se]-OT-OH | 68.1 ± 30.23 | 135 ± 25 |

n.d. . . . not determined
n = 3-6

1. A peptide of formula (I): 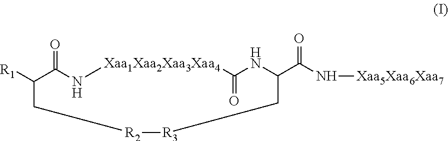
wherein R₁ is hydrogen or NH₂;
R₂ and R₃ are independently selected from —S—, —Se—, —CH2- and —Te—, provided that R₂ and R₃ are not both S or CH₂;
Xaa₁ is L-tyrosine, L-phenylalanine or L-tryptophan;
Xaa₂ is L-isoleucine, D-isoleucine, L-alanine, L-valine, L-leucine or L-methionine;
Xaa₃ is L-glutamine, D-glutamine or L-asparagine;
Xaa₄ is L-asparagine, D-asparagine or L-glutamine;
Xaa₅ is L-proline, D-proline, 4-hydroxyproline or 3,4-dehydroproline;
Xaa₆ is L-leucine, D-leucine, L-isoleucine, L-alanine or L-valine; and
Xaa₇ is absent, glycine, Xaa₈-Xaa₈ or a conservative substitution for glycine;
Each Xaa₈ is independently selected from glycine, or a conservative substitution for glycine; and wherein the C-terminal carboxy group is a free carboxy group (CO₂H) or is —CO₂C₁₋₁₀alkyl or —CO₂C₂₋₁₀alkenyl;
or a pharmaceutically acceptable salt thereof.

2. The peptide of claim 1 wherein Xaa₁ is L-tyrosine.

3. The peptide of claim 1 wherein Xaa₂ is L-isoleucine.

4. The peptide of claim 1 wherein Xaa₃ is L-glutamine.

5. The peptide of claim 1 wherein Xaa₄ is L-asparagine.

6. The peptide of claim 1 wherein Xaa₅ is L-proline.

7. The peptide of claim 1 wherein Xaa₆ is L-leucine.

8. The peptide of claim 1 wherein Xaa₇ is glycine.

9. The peptide of claim 1 wherein the C-terminal carboxy group is —CO₂H.

10. The peptide of claim 1 wherein the peptide is a peptide of formula (II): 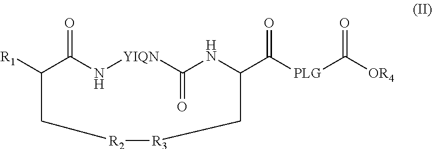
wherein R₁ is hydrogen or NH₂; and
R₂ and R₃ are independently selected from S, Se and Te, provided that both R₂ and R₃ are not S;
R₄ is H, C₁₋₁₀alkyl or C₂₋₁₀alkenyl;
or a pharmaceutically acceptable salt thereof.

11. The peptide of claim 1 wherein both R₂ and R₃ are Se.

12. The peptide of claim 1 wherein the peptide is a peptide of SEQ ID NO:3: 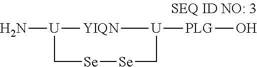
or a pharmaceutically acceptable salt thereof.

13. A pharmaceutical composition comprising a peptide of formula (I) according to claim 1 or a pharmaceutically acceptable salt thereof and a pharmaceutically acceptable carrier or excipient.

14. A method of modulating the oxytocin receptor comprising exposing the oxytocin receptor to a peptide of formula (I) according to claim 1 or a pharmaceutically acceptable salt thereof.

15. A method of treating a condition ameliorated by modulating the oxytocin receptor comprising administering to a subject an effective amount of a peptide of formula (I) according to claim 1 or a pharmaceutically acceptable salt thereof.

16. The method of claim 15 wherein the condition ameliorated by modulating the OTR is a condition in which labor needs to be induced, a condition in which stimulation or reinforcement of uterine contractions is needed, a condition in which milk ejection during lactation is unsatisfactory, sexual dysfunction, depression, anxiety, a anxiety-related condition, schizophrenia, a schizophrenia-related condition, stress, cancer in which oxytocin receptor is over-expressed, gastric disorders, pain or for modifying social relationships.

17. The method of claim 16 wherein modifying social relationships is building relationships in autism.

18. A method of preparing a Se-protected selenocysteine or Te-protected tellurocysteine, suitable for use in peptide synthesis; said method comprising:
i) treating an alanine derivative of formula (III): 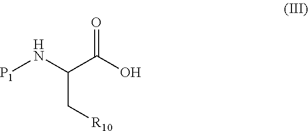
wherein P₁ is hydrogen or an amino protecting group and R₁₀ is a leaving group;
with a diselenide salt or ditelluride salt prepared from combining metallic selenium or metallic tellurium and a first reducing agent in an anhydrous solvent, to produce a diselenide or ditelluride of formula (IV): 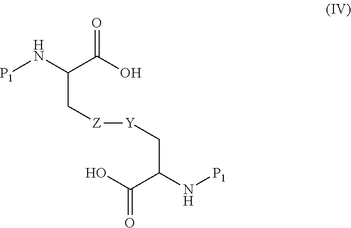
in which Z and Y are Se or Te;
ii) treating the diselenide or ditelluride of formula (IV) with a second reducing agent and an activated protecting group to provide a Se-protected selenocysteine or Te-protected tellurocysteine of formula (V): 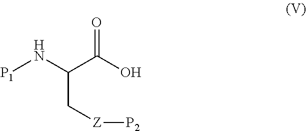
wherein Z is Se or Te; P₁ is hydrogen or an amino protecting group and P₂ is a seleno-protecting group.

19. A method of preparing a Se-protected desamino selenocysteine or Te-protected desamino tellurocysteine, comprising the steps of:
i) treating a propanoic acid with a 3-position leaving group R₁₀ with nucleophilic selenium and to produce a diselenide or ditelluride dimer 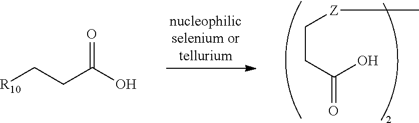
wherein Z is Se or Te;
and reducing the diselenide or ditelluride dimer in the presence of an activated protecting group to obtain the protected 3-selenopropanoic acid 3-telluropropanoic acid
ii)

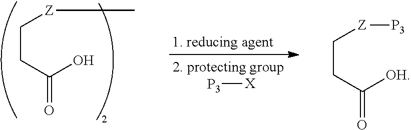

20. A method of preparing a diseleno or ditelluro oxytocin peptide analogue comprising
i) preparing an oxytocin peptide analogue of formula (VI) on a solid phase synthesis resin: 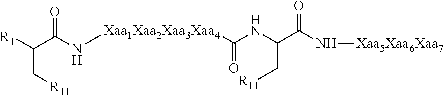
Xaa₁ is L-tyrosine, L-phenylalanine or L-tryptophan;
Xaa₂ is L-isoleucine, L-alanine, D-isoleucine, L-valine, L-leucine or L-methionine;
Xaa₃ is L-glutamine, D-glutamine or L-asparagine;
Xaa₄ is L-asparagine, D-asparagine or L-glutamine;
Xaa₅ is L-proline, D-proline, 4-hydroxyproline or 3,4-dehydroproline;
Xaa₆ is L-leucine, D-leucine, L-isoleucine, L-alanine or L-valine; and
Xaa₇ is absent, glycine, Xaa₈-Xaa₈ or a conservative substitution for glycine;
wherein each R₁₁ is independently selected from Cl, B or I;
ii) treating the peptide with neucleophilic selenium or tellurium to produce a peptide of the formula (VII) 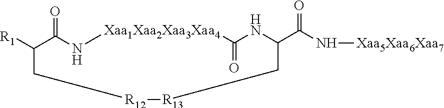
wherein R₁ and Xaa₁ to Xaa₇ are as defined above and R₁₂ and R₁₃ are both Se or Te; and
iii) cleaving the peptide from the solid phase synthesis resin.


SEQUENCE LISTING (33 sequences)

SEQ ID NO: 1 - protein, 9 aa, Artificial Sequence
  Synthetic
CYIQNCPLX

SEQ ID NO: 2 - protein, 9 aa, Artificial Sequence
  Synthetic
CYFQNCPRX

SEQ ID NO: 3 - protein, 9 aa, Artificial Sequence
  Synthetic
XYIQNXPLG

SEQ ID NO: 4 - protein, 9 aa, Artificial Sequence
  Synthetic
XYIQNXPLG

SEQ ID NO: 5 - protein, 9 aa, Artificial Sequence
  Synthetic
XYIQNCPLG

SEQ ID NO: 6 - protein, 9 aa, Artificial Sequence
  Synthetic
CYIQNXPLG

SEQ ID NO: 7 - protein, 9 aa, Artificial Sequence
  Synthetic
XYIQNCPLG

SEQ ID NO: 8 - protein, 9 aa, Artificial Sequence
  Synthetic
XYIQNXPLG

SEQ ID NO: 9 - protein, 9 aa, Artificial Sequence
  Synthetic
AYIQNXPLG

SEQ ID NO: 10 - protein, 9 aa, Artificial Sequence
  Synthetic
XYIQNAPLG

SEQ ID NO: 11 - protein, 9 aa, Artificial Sequence
  Synthetic
XYIQNXPLG

SEQ ID NO: 12 - protein, 9 aa, Artificial Sequence
  Synthetic
XYIQNAPLG

SEQ ID NO: 13 - protein, 9 aa, Artificial Sequence
  Synthetic
XYIQNXPLG

SEQ ID NO: 14 - protein, 9 aa, Artificial Sequence
  Synthetic
XYIQNXPLG

SEQ ID NO: 15 - protein, 9 aa, Artificial Sequence
  Synthetic
XYIQNCPLG

SEQ ID NO: 16 - protein, 9 aa, Artificial Sequence
  Synthetic
CYIQNXPLG

SEQ ID NO: 17 - protein, 9 aa, Artificial Sequence
  Synthetic
AYIQNXPLG

SEQ ID NO: 18 - protein, 9 aa, Artificial Sequence
  Synthetic
XYIQNAPLG

SEQ ID NO: 19 - protein, 9 aa, Artificial Sequence
  Synthetic
XYIQNXPLG

SEQ ID NO: 20 - protein, 9 aa, Artificial Sequence
  Synthetic
XYIQNXPLG

SEQ ID NO: 21 - protein, 9 aa, Artificial Sequence
  Synthetic
XYIQNCPLG

SEQ ID NO: 22 - protein, 9 aa, Artificial Sequence
  Synthetic
XYIQNXPLG

SEQ ID NO: 23 - protein, 9 aa, Artificial Sequence
  Synthetic
XYIQNXPLG

SEQ ID NO: 24 - protein, 9 aa, Artificial Sequence
  Synthetic
XYIQNAPLG

SEQ ID NO: 25 - protein, 9 aa, Artificial Sequence
  Synthetic
XYIQNXPLG

SEQ ID NO: 26 - protein, 9 aa, Artificial Sequence
  Synthetic
XYIQNXPLG

SEQ ID NO: 27 - protein, 9 aa, Artificial Sequence
  Synthetic
XYIQNCPLX

SEQ ID NO: 28 - protein, 9 aa, Artificial Sequence
  Synthetic
XYIQNXPLX

SEQ ID NO: 29 - protein, 9 aa, Artificial Sequence
  Synthetic
XYIQNCPLX

SEQ ID NO: 30 - protein, 9 aa, Artificial Sequence
  Synthetic
XYIQNXPLX

SEQ ID NO: 31 - protein, 9 aa, Artificial Sequence
  Synthetic
CYIQNXPLX

SEQ ID NO: 32 - protein, 9 aa, Artificial Sequence
  Synthetic
CYIQNXPLX

SEQ ID NO: 33 - protein, 9 aa, Artificial Sequence
  Synthetic
CYIQNXPLX